SEQUENCE LISTING (25 sequences)

SEQ ID NO: 1 - DNA, 2010 bp, Saccharomyces cerevisiae
atgagcaatccccaaaaggcactgaacgacttcctcagctccgagagcgtccacacccacgactcctcacgtaagcagtccaacaagcaatcctcggacgagggccgtagctcgagccagccctcccaccatcactccggaggaactaataacaataacaacaataacaacaacaacaacaacagcaacaacaacaacaacggtaacgatggcggcaacgacgacgactatgactacgagatgcaggactaccgtccctccccacagtctgctaggcctactcccacctacgtgccccaatacagtgtcgagtcgggcaccgcattccccattcaagaggtcattccctcggcctacattaacacccaagacatcaaccacaaggacaacggccccccttccgcatcgtcaaaccgcgctttccgcccccgcggccagacgaccgtcagcgccaacgtcctgaacatagaggacttctacaagaatgcggacgatgcacacacaattccagagtcgcatctgagccggcgtcgctcccgatcacgggccacgtcaaatgcaggccactccgccaacaccggcgccaccaatggtcgtactacgggcgcccagaccaacatggagagcaacgagtcccccaggaacgtgccgataatggtcaagcctaagaccctctaccagaacccgcagacccccacagttctcccctcaacttaccacccgattaacaagtggagctccgttaagaatacctatttaaaagagttcctcgccgagtttatgggtacgatggtcatgatcatcttcggcagcgccgtcgtatgccaggtcaacgtcgccggcaagatccagcaggacaacttcaacgtagccctcgacaacctcaatgtcacgggctcttccgccgagacaatcgacgccatgaagagcctgactagcctagtctcctctgtcgccggtggcaccttcgatgacgttgccctcgggtgggccgctgccgtggtaatgggctacttttgtgcaggtggctcggcaatctctggagcccacctgaacccatcaatcaccctcgctaacctcgtttatcgaggctttcccctgaagaaggtcccatattacttcgcaggccagctgattggtgctttcacgggagccctcattctgttcatttggtacaagagggtccttcaggaggcatactcggactggtggatgaacgaatccgtcgctggcatgttttgtgtcttcccaaagccctacctgagctctgggcggcagttttttagcgagttcctctgcggcgccatgctccaggccgggacattcgccctgacggatccctacacgtgcttgagctcagatgtatttcccctgatgatgttcattctcattttcatcattaacgcttcgatggcatatcagacaggtactgcgatgaacctggcacgggatctgggcccccgcctcgccctctacgcagtgggcttcgaccacaagatgctctgggtccaccaccaccacttcttttgggtgccgatggtagggccgtttattggtgccctgatggggggcctcgtctacgacgtctgtatctaccaaggccacgaatcaccggttaactggtctctccccgtgtacaaggaaatgattatgcgcgcatggtttaggcgccccggatggaagaagcggaaccgagctaggaggacttctgacctctccgacttctcttacaataatgacgacgacgaggaattcggcgaacgcatggcgcttcagaagacgaagacgaagtctagcatttccgacaacgagaacgaggccggcgagaagaaggttcagttcaagtccgttcagcgagggaagcggaccttcggcggcatcccgacaattcttgaggaggaggatagcatcgagactgccagcctcggcgccacgacaacggattctattggtctctctgacactagctctgaggacagccactacggcaacgcgaagaaagtcacctga

SEQ ID NO: 2 - protein, 669 aa, Saccharomyces cerevisiae
MSNPQKALNDFLSSESVHTHDSSRKQSNKQSSDEGRSSSQPSHHHSGGTNNNNNNNNNNNNSNNNNNGNDGGNDDDYDYEMQDYRPSPQSARPTPTYVPQYSVESGTAFPIQEVIPSAYINTQDINHKDNGPPSASSNRAFRPRGQTTVSANVLNIEDFYKNADDAHTIPESHLSRRRSRSRATSNAGHSANTGATNGRTTGAQTNMESNESPRNVPIMVKPKTLYQNPQTPTVLPSTYHPINKWSSVKNTYLKEFLAEFMGTMVMIIFGSAVVCQVNVAGKIQQDNFNVALDNLNVTGSSAETIDAMKSLTSLVSSVAGGTFDDVALGWAAAVVMGYFCAGGSAISGAHLNPSITLANLVYRGFPLKKVPYYFAGQLIGAFTGALILFIWYKRVLQEAYSDWWMNESVAGMFCVFPKPYLSSGRQFFSEFLCGAMLQAGTFALTDPYTCLSSDVFPLMMFILIFIINASMAYQTGTAMNLARDLGPRLALYAVGFDHKMLWVHHHHFFWVPMVGPFIGALMGGLVYDVCIYQGHESPVNWSLPVYKEMIMRAWFRRPGWKKRNRARRTSDLSDFSYNNDDDEEFGERMALQKTKTKSSISDNENEAGEKKVQFKSVQRGKRTFGGIPTILEEEDSIETASLGATTTDSIGLSDTSSEDSHYGNAKKVT

SEQ ID NO: 3 - DNA, 996 bp, Trichoderma reesei
atgtcttccggaaggaccgtcaccctcaacaccggctacaagatcccccagatcggctacggcacctggcaggccgctcccggcgaggtcggcgctggtgtctttgaggccctcaaggttggctaccgccacctcgacctggccaaggtctacggcaaccagaaggaggttggtgagggcatcaagaaggctcttgctgaggtccccggcctgaagcgcgaggatattttcatcacctccaagctgtggaacaactcccacaagcccgaggacgtcgagcccgctctcgacgacaccctggccgagcttggcctcgactaccttgacctctacctcatccactggcccgttgcctttgctcccggcgccgacctcttccccaagtccgaggacggctccgaggtgcagctcaaccagaatgtgtccattgtccagacctggaaggccatgaccgagctgcccaagtccaaggtccgctccgtcggtgtctccaactttaccatagagcacctcgacgccgtcatcgaggccaccggcgtcgtccccgccgtcaaccagatcgagcgccacccccgcctccccaaccagcctctgatcgactactgcgccaagaagggcatcatcatcaccgcctactccgcctttggcaacaacacaaagggcctgcccctgctcgtcagctccgacgaggtcaaggccgtcgccgacaacctgtccaagaagcagggcaagaccgtcactcccgcccaggtcatcctcgcctggtcccagattggtggccacaccgtcattcccaagtccgtcaccaaggcgcgcattgcggagaacttccaggaggttgagctggatgacgaggccattgctgcgctgaacaagttgggcgagaagcctcagcggttcaacattccttacacctacaagcctaggtggaacattaacctgttcaacaccgaggaggagaaggccgctgcccacactgctgtcatcaagctgtaa

SEQ ID NO: 4 - protein, 331 aa, Trichoderma reesei
MSSGRTVTLNTGYKIPQIGYGTWQAAPGEVGAGVFEALKVGYRHLDLAKVYGNQKEVGEGIKKALAEVPGLKREDIFITSKLWNNSHKPEDVEPALDDTLAELGLDYLDLYLIHWPVAFAPGADLFPKSEDGSEVQLNQNVSIVQTWKAMTELPKSKVRSVGVSNFTIEHLDAVIEATGVVPAVNQIERHPRLPNQPLIDYCAKKGIIITAYSAFGNNTKGLPLLVSSDEVKAVADNLSKKQGKTVTPAQVILAWSQIGGHTVIPKSVTKARIAENFQEVELDDEAIAALNKLGEKPQRFNIPYTYKPRWNINLFNTEEEKAAAHTAVIKL

SEQ ID NO: 5 - DNA, 2055 bp, Trichoderma reesei
atgggctacacagaggttgaccagaaggccatcaacaccatccgcctgctcgcggtcgatgccaccttcaagagcaactcgggccacccgggtgcccccatgggcatggctcccgttgcccacgtcctttgggacaagttcatgaggttcaaccccaagaaccccaagtggctgaaccgtgaccgattcgttctctccaacggccacggctgcatgctccagtatgctctgcttcacctgttcggctacgacctgaccatcgatgacctcaaggccttccgaaccgtcgacagcaagactcctggtcaccccgaggcccacgatactcccggtgtcgaggtcaccaccggccccctgggtcagggtatctccaatgccgtcggtctggccatggcccaggcccacaccgccgccaccttcaacaagcccggcttcaccctgtgtgacaactacacctactgcttcctcggcgatggctgcttgatggagggtgtctctgccgaggcctcgtccctggccggccacctccagctcggcaacctcatctgcatctacgatgacaaccacatctccattgacggcgacaccaactgtgccttcaccgaggacgtggccaagcgctacgaggcctacggctggcacgtcgaggtcgtcgagaacggcgaccacgacctcgaggccatggaggccgccatcaagaagtgccaggaggtcaaggacaagccctcaatcatcaagctgcgcaccaccattggcttcggctctctccaggagggcacccacggagtccacggctctcccctcaaggccgacgacatcaagcagctcaagcagaagtttggcttcaaccccgacgagagcttcgtcgtccccaaggaggtctccgacctctacgcccgccactcagccgagggcgccgccgccgaggaggagtggaacaagctgttcgccaagtacgccgagaagtaccccgccgaggctgctgacctccagcgccgcctcaagggcgagctgcccgagggctgggagaagaacctgcccgtctactcgcccagcgatgctgccattgccagccgaaagctgtccgagacggtcctcaccaagatccacgacgtcctgcccgagctccagggtggttccgccgatctgactggttccaacctgacccgctggaaggacgctgttgacttccagccccccgccaccggcctcggtgactactccggacgatacatccgctacggtgtccgcgagcacggcatgggcgccatcatgaacggcctggccgcctacggcaccatcatcccctacggcggtaccttcctcaacttcgtctcctacgccgccggtgccgtccgcctgtccgctctgtcccgcgtccgcgtcatctgggtcgccacccacgactccattggcctgggcgaggacggtcctactcaccagcctatcgagactctggctcacttccgcgccctgcccaactgcatggtgtggcgccccgccgacggcaacgagaccagcgctgcctactacattgccctgacctccaagcacacccccagcatcattgccctgtctcgccagaacctgccccagctcgagggctccgtcatcgagaaggccatcaagggaggatacgtcctgcaggaggtcgagggtgcccagatcaccctggtctccaccggttccgaggtttccatcgccgtggatgccgccaagtacctcctggagaagcacaacgtcaaggcccgcgtcgtctccatgccctgcttcgaggttttcgatgctcagcccaaggagtacagactctccgtcctgcccgacggcatcccctctctgtccattgaggtgatgagcaccatgggctgggagcgatacactcacgagcagtttggtctcaaccgattcggtgcctctggcgcctacaaggatgtctacaagaagttcgagttcacccccgagggcatcaccaagcgcgctcttctcaccattgacttctggaaggacgtccccaacgtccgctctcccatcaaccgtgccttccagcagctcatc

SEQ ID NO: 6 - protein, 685 aa, Trichoderma reesei
MGYTEVDQKAINTIRLLAVDATFKSNSGHPGAPMGMAPVAHVLWDKFMRFNPKNPKWLNRDRFVLSNGHGCMLQYALLHLFGYDLTIDDLKAFRTVDSKTPGHPEAHDTPGVEVTTGPLGQGISNAVGLAMAQAHTAATFNKPGFTLCDNYTYCFLGDGCLMEGVSAEASSLAGHLQLGNLICIYDDNHISIDGDTNCAFTEDVAKRYEAYGWHVEVVENGDHDLEAMEAAIKKCQEVKDKPSIIKLRTTIGFGSLQEGTHGVHGSPLKADDIKQLKQKFGFNPDESFVVPKEVSDLYARHSAEGAAAEEEWNKLFAKYAEKYPAEAADLQRRLKGELPEGWEKNLPVYSPSDAAIASRKLSETVLTKIHDVLPELQGGSADLTGSNLTRWKDAVDFQPPATGLGDYSGRYIRYGVREHGMGAIMNGLAAYGTIIPYGGTFLNFVSYAAGAVRLSALSRVRVIWVATHDSIGLGEDGPTHQPIETLAHFRALPNCMVWRPADGNETSAAYYIALTSKHTPSIIALSRQNLPQLEGSVIEKAIKGGYVLQEVEGAQITLVSTGSEVSIAVDAAKYLLEKHNVKARVVSMPCFEVFDAQPKEYRLSVLPDGIPSLSIEVMSTMGWERYTHEQFGLNRFGASGAYKDVYKKFEFTPEGITKRALLTIDFWKDVPNVRSPINRAFQQLI

SEQ ID NO: 7 - DNA, 975 bp, Trichoderma reesei
atgagctcctctcttgaccagctcaaggccactggcaccgttgtcgtctctgacactggtgactttgctgccatcgccaagtacaagccccaggatgccaccaccaacccctccctcatcctggccgcctccaagaagcccgagtacgccaagctgatagacgtcgccatcgactacgccaagcagaagggcggcgacattgagcagcaggtcgacgatgccctcgaccgcctgctggtcgagttcggcaaggagattctcaagattgttcccggcaaggtctccaccgaggtcgacgcccggttctcctttgacaccaaggcctctgtcgacaaggccctccacattatcgagctctacaaggagctcggcatccccaaggagcgcgtcctcatcaagatcgcctccacctgggagggcatcaaggccgccgagatcctgcagcgcgaccacggcatcaactgcaacctgacgctcatgttctccctgccccaggccatcggcgccgccgaggccggcgccttcctcatctcccccttcgttggccgcatcctcgactggttcaaggcgagcaccaagaaggactactccaaggaggaggaccccggtgtcgcctccgtcaagagcatcttcaactactacaagaagtttggctacaagaccattgtcatgggtgcctcgttccgcaacacgggcgagattaccgagctcgctggctgcgactacctgaccatttctcccaacctgctcgaggagctcctcaactccaccgagcccgtccccaagaagctcgacgccgccgccgctgcctcccttaacattgagaagaagtcgtacatcaacgacgagtccctgttccgcttcgacttcaacgaggaccagatggccgtcgagaagctgcgcgagggcatcagcaagtttgccgccgacgccgtcaccctcaaggacattctgaggcagaagctgtctgcttaa

SEQ ID NO: 8 - protein, 324 aa, Trichoderma reesei
MSSSLDQLKATGTVVVSDTGDFAAIAKYKPQDATTNPSLILAASKKPEYAKLIDVAIDYAKQKGGDIEQQVDDALDRLLVEFGKEILKIVPGKVSTEVDARFSFDTKASVDKALHIIELYKELGIPKERVLIKIASTWEGIKAAEILQRDHGINCNLTLMFSLPQAIGAAEAGAFLISPFVGRILDWFKASTKKDYSKEEDPGVASVKSIFNYYKKFGYKTIVMGASFRNTGEITELAGCDYLTISPNLLEELLNSTEPVPKKLDAAAAASLNIEKKSYINDESLFRFDFNEDQMAVEKLREGISKFAADAVTLKDILRQKLSA

SEQ ID NO: 9 - DNA, 9538 bp, artificial sequence
  pKR_ReAsl1fps1
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgagtgaaggccgtcaaggcctaggcgcgccagcggccgcgaaatagaagacgaagacgagaggaggagagggtgcgagagggtgcgaaactgcgcccttcagctcgctaattccgcccattacattcagcgcacacttggtggtacatacatacctacatacgtacccgtagttgtgcgaagcgagtcgagccagcggtcggatgtgccaatatgtaccttgtaggtactcatgatagtcagcaggaagccgagggatgatagcacgcacagccagtctggtgtttgtgctctggattcagcccctagattcactgaccaaaagaagcaagagaagggaaagggaaaaggaagccctaaaaaagagcagggttgtccttcggcaccaccccgaaagcaacagcagtgatggacgggactacgacactgtagtacatacgccaacccaaagattccgagttgcaatcattgccacaaaattgctcgactgtggcgggagaggggaaagtctccgcgagggcatctctgatgagtcccgtgccacgccgggtttcttgccttgaatcaccacaaaatgtctcagcgcggctcatccatggccgcctttttgggcgtttcgtattactcttgggtttgactgtattatcggtcaagagccaaaggccactcggcttttccgcccttgcctctcttccatttccccaggcgctgatagtatgatagtttcccctcattgcagctttggacgtcaatgccaataactgtattgtattgacaaatcatttgtccaaagacgaccgacacctctgatatcacatacgagtagtagtacatacaatcccccccaaactccgcctttttggcgcgtgtgtgccgcgtagctcgtgcacaatgtcgggccgccgccgccagcggccacttgcttagcaggaacctagtcagccgtccttgtgtgattaatataacggtgagactagcggccggtcccttatccagctgtccacgttggcctgccctcagttagcgctcaactcatgccctcactggcgaggcgagggcaaggatggaggggcagcatcgcctgagttggagcaaagcgccgccatgggagcagcgaaccaacggagggatgccgtgctttgtcgtggctgctgtggccaatccgggcccttggttggctcacagagcgttgctgtgagaccatgagctattattgctaggtacagtatagagagaggagagagagagagagagtgtgagagggaaaaggtgaggttgaagtgaggtagtaagtgaaaaaaaaaaaaaaatcaacactgacggctgccgtctgccaccctcctccacccagccacctgcacactcagcgcgcagcatcacctaatcttggctcgccttccgcagctcaggttgttttttttttttctctctcctcgtcgaagccgcccttgttcccttatttatttccctctcctaccttgtctgcctttggtccatctgcccctttgtctgcatctcttttgcacgcatcgccttatcgtcgtctcttttttcactcacgggagcttgacgaagacctgactcgtgagcctcacctgctgatttctctccccccctcccgaccggcttgacttttgtttctcctccagtaccttatcgcgaagccggaagaaccctcttaacctctagaatgagcaatccccaaaaggcactgaacgacttcctcagctccgagagcgtccacacccacgactcctcacgtaagcagtccaacaagcaatcctcggacgagggccgtagctcgagccagccctcccaccatcactccggaggaactaataacaataacaacaataacaacaacaacaacaacagcaacaacaacaacaacggtaacgatggcggcaacgacgacgactatgactacgagatgcaggactaccgtccctccccacagtctgctaggcctactcccacctacgtgccccaatacagtgtcgagtcgggcaccgcattccccattcaagaggtcattccctcggcctacattaacacccaagacatcaaccacaaggacaacggccccccttccgcatcgtcaaaccgcgctttccgcccccgcggccagacgaccgtcagcgccaacgtcctgaacatagaggacttctacaagaatgcggacgatgcacacacaattccagagtcgcatctgagccggcgtcgctcccgatcacgggccacgtcaaatgcaggccactccgccaacaccggcgccaccaatggtcgtactacgggcgcccagaccaacatggagagcaacgagtcccccaggaacgtgccgataatggtcaagcctaagaccctctaccagaacccgcagacccccacagttctcccctcaacttaccacccgattaacaagtggagctccgttaagaatacctatttaaaagagttcctcgccgagtttatgggtacgatggtcatgatcatcttcggcagcgccgtcgtatgccaggtcaacgtcgccggcaagatccagcaggacaacttcaacgtagccctcgacaacctcaatgtcacgggctcttccgccgagacaatcgacgccatgaagagcctgactagcctagtctcctctgtcgccggtggcaccttcgatgacgttgccctcgggtgggccgctgccgtggtaatgggctacttttgtgcaggtggctcggcaatctctggagcccacctgaacccatcaatcaccctcgctaacctcgtttatcgaggctttcccctgaagaaggtcccatattacttcgcaggccagctgattggtgctttcacgggagccctcattctgttcatttggtacaagagggtccttcaggaggcatactcggactggtggatgaacgaatccgtcgctggcatgttttgtgtcttcccaaagccctacctgagctctgggcggcagttttttagcgagttcctctgcggcgccatgctccaggccgggacattcgccctgacggatccctacacgtgcttgagctcagatgtatttcccctgatgatgttcattctcattttcatcattaacgcttcgatggcatatcagacaggtactgcgatgaacctggcacgggatctgggcccccgcctcgccctctacgcagtgggcttcgaccacaagatgctctgggtccaccaccaccacttcttttgggtgccgatggtagggccgtttattggtgccctgatggggggcctcgtctacgacgtctgtatctaccaaggccacgaatcaccggttaactggtctctccccgtgtacaaggaaatgattatgcgcgcatggtttaggcgccccggatggaagaagcggaaccgagctaggaggacttctgacctctccgacttctcttacaataatgacgacgacgaggaattcggcgaacgcatggcgcttcagaagacgaagacgaagtctagcatttccgacaacgagaacgaggccggcgagaagaaggttcagttcaagtccgttcagcgagggaagcggaccttcggcggcatcccgacaattcttgaggaggaggatagcatcgagactgccagcctcggcgccacgacaacggattctattggtctctctgacactagctctgaggacagccactacggcaacgcgaagaaagtcacctgaatgcatggctttcgtgaccgggcttcaaacaatgatgtgcgatggtgtggttcccggttggcggagtctttgtctactttggttgtctgtcgcaggtcggtagaccgcaaatgagcaactgatggattgttgccagcgatactataattcacatggatggtctttgcgatcagtagctagtgagagagagagaacatctatccacaatgtcgagtgtctattagacatactccgagaataaagtcaactgtgtctgtgatctaaagatcgattcggcagtcgagtagcgtataacaactccgagtaccagcaaaagcacgtcgtgacaggagcaggctttgccaactgcgcaaccttgcttgaatgaggatacacggggtgcaacatggctgtactgatccatcgcaaccaaaatttctgtttatagatcaagctggtagattccaattactccacctcttgcgcttctccatgacatgtaagtgcacgtaggaaaccatacccaaattgcctacagctgcggagcatgagcctatggcgatcagtctggtcatgttaaccagcctgtgctctgacgttaatgcagaatagaaagccgcggttgcaatgcaaatgatgatgcctttgcagaaatggcttgctcgctgactgataccagtaacacctttgcttggccgtctagcgctgttgattgtattcatcacaacctcgtctccctcctttgggttgagctctttggatggctttccaaacgttaatagcgcgtttttctccacaaagtattcgtatggacgcgcttttggctgtattgcgtgagctaccagcagcccaattggcgaagtcttgagccgcactcgcatagaataattgattgcgcatttgatgcgatttttgagcggctgtttcaggcgacatttcgcccgcccttatttgctccattatatcatcgacggcatgtccaatagcccggtgatagtcttgtcgaatatggctgtcgtgaataacccatcggcagcagatgataatgattccgcagcacgctagcgagctctccggagcccaaaatagaagcacccgaattcagggcctttttctccacaccaatcaccaaattcactgatttcaaggttgttagccgcctcttatcactaggtagactagcacctaattttaccgatatgtgtagtccatacatgtatgagatatacatcatgatctcgaagtaaaacccggtacgaagaggtataaaagccgcggaacaacaacttgcagatgccatcacgcaagacgcagacacggctctcttctcgcgggcatcaccagagcccaatcacctggtcctcaggtggccacttgagcgccgtcagaatggccgcacagcagcagcagcatcaccacagatcttggcctcaactcttgatatttcggcgagaatacaattactagtctagtctatggaaattggatgttgtgcggctctttggagactggtagtacataagccacgaacatgatattggcggggaaggtttagctggaaattggaacaagtcagagaagtcgccgcgtattagcttggtgttagtagacacagcaagtcaagagtgggagttagatttctgaattgagtgacgcagatggatgtagtattcattattgaagacagagcctttccatcaacgtgcagagccatgaaatgcaatgaccttaaggaggggcatctctccgctttccatctgactgagatactaacccgattccatctccattttctagcacacacatacctggagtcatacctcaaccggccttcagtagctaaacagctatgtagcgcatccgcgctaagccgcttaggcgataacgttggccggtgtcggccatacaattcttgcctatgactctgtctcgactaactgattgaccaacgtcaattccttatcaaagttttcgaccttgataactctcgatcttaatctcgagctttttatttcctcaacatcattcacagtaacgactcaagcccttggacttggccagcaattgacacaaccacgcgccatctgctccattggcctcatcgatcccatcgacttcgtcagcctcgacaacgcatccaacgcaatcacaaatacttcaacatggctgagaaaaaggcggacgccccggcagccggcatgctctggggtggcaggttcacaggttgcagcctcgcaaatctagctgcggattcagtatactgacctctcgcgatgcgcaggtgcaattgatcctctcatgcacaagtacaatgcctccatcgggtacgacaaggccctgtacaaggaagatatccttggatccattgcctttgcgagggccaattcaaaggctggcatcatcagcgaagacgaattccaggcgatcgagcgtggcttgctccaagtgatggaagaatggaagcagggcactttcgccatcatgccgaatgacgaagatgtgagagggcacactcgattctaatctcaaacttctttcgaggctgacacaatggaccagattcatacggcaaacgaacgtcgcctgggagaggtcatcggcaaggacatcgcgggaaagcttcacacaggtcgcagccgcaacgagcaagttgtctgtgatatgcgcatgtggcttcgcgatcggatccgcgaaatcgacagccaactcgttgctttcctcaaggtcattatcacacgtgccgaatctgaaatgtaagcccagccgtacctttccagctaaatgccccccccccccccccccccctctcttactgacggacaacctcagcgactacatcatgcccggatacacccatctccagcgcgctcagccagttcggtggtctcaatggctcatgtcctatgccgctgcgttcaagcaggatctcgagcggttgcgtcaagtcttcgagagggtcaacctcagtccacttggctgtggcgccttggccggcaatccattcggaatcgaccgagactcaatagctgcggagcttggcttcagcggaatcaccttgaactccatgaatacttcggccgaccgcgatttcctcgtcgatttcctgtcctggaactccatcttcaccaaccacgtcagcaggtgggctgaggacctcatcatctactcgacctctgagttcagtttcgtccgtctggccgacgcctattccacgggcagctctctcatgcccaacaaattcaacccggactcgttggagcttcttcgcggcaagtcagggcgtgctttcggccaaatggctggtttgatgatggtgagtcctcatatcaaacatctcagccagtttgacttggcttttgagggtgaccgcggccgccgttccggtaattaactggcctcatgggccttcacttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattatttacccaccactttggtaatttcgcctttcacgtaatgcacaaattcttccagctgatccgcacggctggccagacgatcttcttcctggcccagatacgcctgacgcgcttccagaatcaccggctgatactgcgccggcagacgttccatggcccaatccgccgccacatctttcggcgcaattttgccggtcaccgcgctataccaaatacggctcagggtcagcaccacgttgcgttcatcacccgcccaatccggcgggctgttccacagggtcagggtttcgttcagcgcttcaaacaggtcctgttccggcaccggatcaaacagttcttccgctgccggaccaaccagcgccacgctatgttcacgcgctttggtcagcagaatggccagatcgatatcaatggtcgccggttcaaaaatgccggccagaatatcgttacgctgccattcgccaaactgcagctcacgtttcgccggataacgccacggaataatgtcatcatgcaccacgatggtcacttccaccgcacgcagaatttcgctttcacccgggctcgcgctggtttccagcagatcgttaatcagcgcacgacgggtggtttcatccagacgcacggtcacggtcaccagcagatcaatatcgctatgcggtttcaggccgccatccaccgcgctgccatacagatgcacggccagcagggtcggttccagatgacgttcaatcacgcccaccacttcagacagctgggtgctcacttccgcaatcaccgcttcacgcataatgttcagtttggttttcggacggctgccctgctgggtcacatcgttgctgctccacagcatcaggcaacggcccaccgcataacgcgcctgctgtttgctcgcacgcggcatgctctgcacgccaaaaaagcaatcatagcacgccataaacaccgccaccgcaccattaccgccgctacgttcggtcagggtacggctccaattacggctacgcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 10 - DNA, 11413 bp, artificial sequence
  pKR_Rpyr4err1
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgagtgaaggccgtcaaggcctaggcgcgccagcggccgccagatctcgagatagtatctcattcagtaaaatactcgtacgagtttgagggggcgcgaggctaagctgcaagcgggcgcggcaacactacctgtatgtattaataaagcagcagagagagagagagccctggactgccggcaatcctgagaaggggggatcgaccggaccatctgcacgcagcagggctgctgctgccaacatggagagactaaagatgggattggtggtacatagcaagacgcttggatgcttgttgacgggctgctccaataagcaagctcacaagcagtgctttgggggtccgcacggacggtcgagagggctcggacggctgatattgcagacctggacgagtcagttgtgcaggtaggcactcgtatgtgtgtctttgtgcttgatcaaccttgagtaagtattattagggtgatggcttaggtaggtaggttaggtaggtagtttaggtacagggaggtgaggaggggagacgtccagctgccatatgcgccacttgcagcctcaactctgcaaaagccagccccgatcacggcatgtcgtacatactgtaaggtagtacctagtagtagaacgaatgctaaaggtgcagagcaactaacttgagcaaaaccccccctccccccatcgagattgattgccctctacgctgaaaatagtctcgccagccggcgacgcttagccgtggcgctaccgcgacatggcgcttgttagcctgcggtccaggttgatgcgaagcatgtcgcagggcgggtctcgtgcgtgccgacagccagcagagagacagaagagagcgagagagcgagagagcgagagagagagagagagagaaagacgcctcgggggtttggctggtggcttcgatgcttgtccgtgtcaagggttcacgaaagacgggacggctgagttggtgactggcggcggtggcatcgaggctggagtccaggcacatgcatggcgcaaggcatgtcaggccatgtcgtgtcatgccccggcacagctgaagggaatactactattgagagctgttctccaaggcgtcaagcatgtgagcaaaaactcgagttgtctagactcgactcgacttgcggctgcagtacaacaatacagtactactcgtacacgtatgtacagcaaggagcaggtggatcagcaaagttgcatgataatggactggaccggcaacgctcaaagttcgccttgcagctccggcatgaagggcagacccccgccattattagcgatgactagccgtggaggccactttggctttagtgatttaaaccccgagattcatgctcgatgctctacaggtatgtatgtactagctcagctcagccttagtaggtaggtattgaagtctggtggtgcgaggaggacctgcaggtgggagttttgggggaaaaagatgatgctcaccaactaaaataaatagtcaggccacacacacaacctactgagcagaaccaccacaaccagtgaagagctactccggaattctcgactccggagagttgggcaaaatcaggcgcggaccagcgccaaggtgtgggtggattgggtcgtaagaaaaagaggcatccctacatgcagcaaccggctggagacgagccagcccgagcactggcagcctcagaccagaggcatctcgaccacggcttcaagtatcagactgggagctgtgccgagtttgctggctacttacctagtcaaaaaggtcgccgctgactgtctgtgtcatattaacgagccaggtctcaaccttctgtcgaatgaaatggaaagcggcgaatgactcttcggggttggccagaaacaacgacgaccactgagggatcgggacgatgtacatctactgcgatatgccgggctcgaatgtgactgcacacaccacacacacacacacacacggacacatctgagcagtactatcaaagcggtgggagtgcatctgcactgggcacccttactgtaggtaggaaggatacctaaggtagagggctcgctgctgcaagttgatatgacggccaccaatcggaagggtgtcgtatctcacatcaccagcgtcaggacacgcttcacaaaattcccacaagttccagttgaaatgacccgtaggagagcgcctcgagggacagaatgtacagtactatactacctaccttagatgccatcaccccttcgcaggccatggaatttctggcaagtcgcacccttcagccatgcctgtccgccatcatcataccagatcgtgtcgtggcgggcacttgggaaggacgagctccaaccgccgccgctgattggcctgatccgccacagccactggcgctcgcggcgaggaaaaaaaaaaatcttgcgggtgtgcagcagcattgagtggaaagcgggagccgctgcacgacgctgattcgtccgggctgcctcgttagggctagaccagcaggcgggcgccgcttattttgcccctcctcaggctttttttcacaagccccgttttgctgccccgcgcgtggcagaaaaaaaaaatacagcacgagccactggcacttgcacccctccacccctccgccgaccaccttttaaatctcctcctcccaccatctgctttcgaaattttctctcttctgctcttcgtctcgcatacccggttcaagcatccgatctgcgaatttgtatgtgctgcctctccctctgaccttctggtctggtgataccatcctccctcagtttggatcatcgccttattcttcttccctcttctgcatctgcttcctgctcgtttgaggaacatcgccagctgactctgcttgcctcgcagcgatctagtcaagaacaacacagctctcacgctacatcacacttaagtctagaatgtcttccggaaggaccgtcaccctcaacaccggctacaagatcccccagatcggctacggcacctggcaggccgctcccggcgaggtcggcgctggtgtctttgaggccctcaaggttggctaccgccacctcgacctggccaaggtctacggcaaccagaaggaggttggtgagggcatcaagaaggctcttgctgaggtccccggcctgaagcgcgaggatattttcatcacctccaagctgtggaacaactcccacaagcccgaggacgtcgagcccgctctcgacgacaccctggccgagcttggcctcgactaccttgacctctacctcatccactggcccgttgcctttgctcccggcgccgacctcttccccaagtccgaggacggctccgaggtgcagctcaaccagaatgtgtccattgtccagacctggaaggccatgaccgagctgcccaagtccaaggtccgctccgtcggtgtctccaactttaccatagagcacctcgacgccgtcatcgaggccaccggcgtcgtccccgccgtcaaccagatcgagcgccacccccgcctccccaaccagcctctgatcgactactgcgccaagaagggcatcatcatcaccgcctactccgcctttggcaacaacacaaagggcctgcccctgctcgtcagctccgacgaggtcaaggccgtcgccgacaacctgtccaagaagcagggcaagaccgtcactcccgcccaggtcatcctcgcctggtcccagattggtggccacaccgtcattcccaagtccgtcaccaaggcgcgcattgcggagaacttccaggaggttgagctggatgacgaggccattgctgcgctgaacaagttgggcgagaagcctcagcggttcaacattccttacacctacaagcctaggtggaacattaacctgttcaacaccgaggaggagaaggccgctgcccacactgctgtcatcaagctgtaaactagtgaattctggatcctttcgtgaccgggcttcaaacaatgatgtgcgatggtgtggttcccggttggcggagtctttgtctactttggttgtctgtcgcaggtcggtagaccgcaaatgagcaactgatggattgttgccagcgatactataattcacatggatggtctttgcgatcagtagctagtgagagagagagaacatctatccacaatgtcgagtgtctattagacatactccgagaataaagtcaactgtgtctgtgatctaaagatcgattcggcagtcgagtagcgtataacaactccgagtaccagcaaaagcacgtcgtgacaggagcaggctttgccaactgcgcaaccttgcttgaatgaggatacacggggtgcaacatggctgtactgatccatcgcaaccaaaatttctgtttatagatcaagctggtagattccaattactccacctcttgcgcttctccatgacatgtaagtgcacgtaggaaaccatacccaaattgcctacagctgcggagcatgagcctatggcgatcagtctggtcatgttaaccagcctgtgctctgacgttaatgcagaatagaaagccgcggttgcaatgcaaatgatgatgcctttgcagaaatggcttgctcgctgactgataccagtaacacctttgcttggccgtctagcgctgttgattgtattcatcacaacctcgtctccctcctttgggttgagctctttggatggctttccaaacgttaatagcgctagtagtagaaatggagggtaaactgaacatcagtcaaaagacgtcgagacgcagatactactagatacctatcccagcgacggaaaatggacaaggtgtcgccgccgccaagctctcagatgcgctatcgtgcatgatgcctctcatcaacaaacggcgccagtgacgggctagccgtgggattgactcgagctcgcctcttctttgtgcttttctcctgtcgacatcccggcttgcgcttggacctcgccgggacgtgcaaacgaacacatcactttcaaagagagtgtttgatgctcacgctcggatgctccagcccccccttgcttgcttgtccatcattgaggctcgaaggggcagttgctccccattccctctgctccaacaggaactgtaaccgaaaaaaaagccatcctccttcttcctcttcctcttccgcctcctcctcttcctcctcctactaatacctactcctcctctcctcatcgactagactgacccccccggttgggcccctcgtcccgtctccaacagagcaccaccagacaaagacccctgcccgcgcgaatccagacccccccagcaattccgggcctcgttgatcctcctctactgtagttgtacatacatacctaccgactgcattgcattggtacagctgcaggcacttccaggcacggccaccaaattgcagcggcccttgcttgcttgcttggttcgagaactaggatctgtgtcttttgccttgccttgtcttgtctgggttcctgctcgtctgcggcaatcggaacgccgccagtgcggtgccaagcaaaccagccaaggtaggtacctaccactaggcttcttttctcgttgtctcactctctcttttcctctttgtcctctcttatccccatcttttctctctctctctgctcctttcctaaccacttccctacctttctctttttccttttcttgtcatctccatcttggctgacgaaaaaggtctgactgggtaggtattatctggcagacttgtgtgtatcattcaccctatttctgcttcatagtacatgtactgtacctgaacggctcaaccgctatttacgactcttatttttttgtggcgttggtcacgtttgccagctgttgtccgtctttctagggctcctcaaacttgacctgaccgagctccctttctggacccggtgggcttcacttccagctgctgagcgacctgagccgaacatcctcagtccttgtccagcgcaattcattttctttccttttctttttttttattcctttctttacttttattctctctttttctcctcttcctcttcttcttctttctcctcctcctccatatcctcactctcgtctccctcattactaccctctcggctcctcaggtccaccaaccctcccgcacccaaacctctgccgctgaaacccattcggtggtcgccgtttttttttttttttttttctcacccccaaagtcgcaatatcgggtatcgccgccggcattgaatcgccttctccgctagcatcgactactgctgctctgctctcgttgccagcgctgctccctagaattttgaccaggggacgagcccgacattaaagcaactccctcgcctcgagacgactcggatcgcacgaaattctcccaatcgccgacagttcctactcctcttcctcccgcacggctgtcgcgcttccaacgtcattcgcacagcagaattgtgccatctctctcttttttttccccccctctaaaccgccacaacggcaccctaagggttaaactatccaaccagccgcagcctcagcctctctcagcctcatcagccatggcaccacacccgacgctcaaggccaccttcgcggccaggagcgagacggcgacgcacccgctgacggcttacctgttcaagctcatggacctcaaggcgtccaacctgtgcctgagcgccgacgtgccgacagcgcgcgagctgctgtacctggccgacaagattggcccgtcgattgtcgtgctcaagacgcactacgacatggtctcgggctgggacttccacccggagacgggcacgggagcccagctggcgtcgctggcgcgcaagcacggcttcctcatcttcgaggaccgcaagtttggcgacattggccacaccgtcgagctgcagtacacgggcgggtcggcgcgcatcatcgactgggcgcacattgtcaacgtcaacatggtgcccggcaaggcgtcggtggcctcgctggcccagggcgccaagcgctggctcgagcgctacccctgcgaggtcaagacgtccgtcaccgtcggcacgcccaccatggactcgtttgacgacgacgccgactccagggacgccgagcccgccggcgccgtcaacggcatgggctccattggcgtcctggacaagcccatctactcgaaccggtccggcgacggccgcaagggcagcatcgtctccatcaccaccgtcacccagcagtacgagtccgtctcctcgccccggttaacaaaggccatcgccgagggcgacgagtcgctcttcccgggcatcgaggaggcgccgctgagccgcggcctcctgatcctcgcccaaatgtccagccagggcaacttcatgaacaaggagtacacgcaggcctgcgtcgaggccgcccgggagcacaaggactttgtcatgggcttcatctcgcaggagacgctcaacaccgagcccgacgatgcctttatccacatgacgcccggctgccagctgccccccgaagacgaggaccagcagaccaacggatcggtcggtggagacggccagggccagcagtacaacacgccgcacaagctgattggcatcgccggcagcgacattgccattgtgggccggggcatcctcaaggcctcagaccccgtagaggaggcagagcggtaccgatcagcagcgtggaaagcctacaccgagaggctgctgcgataggggagggaagggaagaaagaagtaaagaaaggcatttagcaagaagggggaaaagggagggaggacaaacggagctgagaaagagctcttgtccaaagcccggcatcatagaatgcagctgtatttaggcgacctctttttccatcttgtcgatttttgttatgacgtaccagttgggatgatggatgattgtaccccagctgcgattgatgtgtatctttgcatgcaacaacacgcgatggcggaggcgaactgcacattggaaggttcatatatggtcctgacatatctggtggatctggaagcatggaattgtatttttgatttggcatttgcttttgcgcgtggagggaacatatcaccctcgggcatttttcatttggtaggatggtttggatgcagttgtcgacgatatcagcttccatatatacggaattgttcaattgagtattacctgatggctatttgtgtcgatcctttgtgacattgacatgtgatggcgcctgtacctctaaatgtatgcagcgtaacatcttcagagcacggcggcatgttctcccctctacaatattcagataagcagtacttgtgctgtgagaaacccattgatcgctggtggcaaccaaggcgatatgtctccaggagaggaaattgcacgatcattgatcgtttgtgctactcgtttcgtgtccaagcgaacagagtattaatatataccattatatcatatgcatcgtggagtcgttactatgcagcctaacctaaggtacctacaaatcggcatcccggagttaacgcggaggctctcatcgccgggcgtctccaccaattcgacatatcaatgctaatcgggcgcaaaggctccaacgcggatgctggtcgcattgaggttccgcattccttttcctccttccgaccctctttcttacatctcactctcacatcccgtttgccggcccgctccccaccgagacacgaaacggacggcaacaatcaaccgtccccccggaacagcgacctgacctcccctccccccatatacgattacgagctgaacgagacgagagagagagagagagagaagaagaagacagagaagaggcatggccgacattgaggtggcagtcaacgtacgggccgagcggggcccgactaagaagcggagacggatcgtcatctcgtgcaccgagtgccatcgaaggaagcaaaaggtcaggcttctttcctaccaagaaacaaagtgaagcttattaattaactggcctcatgggccttcacttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattatttacccaccactttggtaatttcgcctttcacgtaatgcacaaattcttccagctgatccgcacggctggccagacgatcttcttcctggcccagatacgcctgacgcgcttccagaatcaccggctgatactgcgccggcagacgttccatggcccaatccgccgccacatctttcggcgcaattttgccggtcaccgcgctataccaaatacggctcagggtcagcaccacgttgcgttcatcacccgcccaatccggcgggctgttccacagggtcagggtttcgttcagcgcttcaaacaggtcctgttccggcaccggatcaaacagttcttccgctgccggaccaaccagcgccacgctatgttcacgcgctttggtcagcagaatggccagatcgatatcaatggtcgccggttcaaaaatgccggccagaatatcgttacgctgccattcgccaaactgcagctcacgtttcgccggataacgccacggaataatgtcatcatgcaccacgatggtcacttccaccgcacgcagaatttcgctttcacccgggctcgcgctggtttccagcagatcgttaatcagcgcacgacgggtggtttcatccagacgcacggtcacggtcaccagcagatcaatatcgctatgcggtttcaggccgccatccaccgcgctgccatacagatgcacggccagcagggtcggttccagatgacgttcaatcacgcccaccacttcagacagctgggtgctcacttccgcaatcaccgcttcacgcataatgttcagtttggttttcggacggctgccctgctgggtcacatcgttgctgctccacagcatcaggcaacggcccaccgcataacgcgcctgctgtttgctcgcacgcggcatgctctgcacgccaaaaaagcaatcatagcacgccataaacaccgccaccgcaccattaccgccgctacgttcggtcagggtacggctccaattacggctacgcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 11 - DNA, 4768 bp, artificial sequence
  mpdh deletion cassette
aaagacatgaaccaggctggagacgagatagtcaggcaccagatggcgagatgggacgaggaagaaccatggacgaggcgaagaatgaggcaaagttggaatgacggactgcagtgagtggatggagttcaaagtacctgggtacctacatactgcgtgcatgggccacgataccggcaaggaccagcgacacggcaggtacgaagcacgaaccagcaagcacgaaccagcatcgtccgtaaatgcaggtgcgttcctgccttctagacggcggtaccagataagagacgctgcaccgcacataacggaaggtcactcgacccgctttcaaggtggctcttgcgccacggcctgctggaagccacgccccatgattggccagcagagaggctcatgccactggacagctgtactggtacctgctgcttctcacctcatgccctgatgcgttgttggactgatgggatggtctcacccgtacgacgtagcacgaggcctggaccactctggcattgacacggcactcaatgcaacaatggcatccgatggccgctgtatgttgtatcatgtagagcatcgagtacttccactcgtacctgtacctgcgctctcgaatggggaccccccctgtggatctggcggtacaagctgcatgtactccctggactcggtccacaccccccctgctgtgacgatggtcgatcagcttcaaagtccctccatcttcttcgcctgttggacccgattgaggcgccatcgtcatcgggaccccctacctgacctctggcctgtaatactgtcctggctgtatgtcctgctaccacctggagcagcctttcggtggctctcgtgcttctcgtggactctgtcaaactcatcctcgaattattataaagctggccggactcccgctcctcctacttgactcttagttcctctccatcatcaaccagctttcaccacacaaaaactcccatttctctttctctttctcttcctcctacaaccaccacatcgccaactcttccttctgcaactccaactccttcacttctcttctctgttctactcttctccacctacttcacctaccgcaatcgagagctaccttacatcaatatggccagcacctcttcggcgatacatactcgccaccccagccggggcgattgtgtgtactaggtaggctcgtactataccagcaggagaggtgctgcttggcaatcgtgctcagctgttaggttgtacttgtatggtacttgtaaggtggtcatgcagttgctaaggtacctagggagggattcaacgagccctgcttccaatgtccatctggataggatggcggctggcggggccgaagctgggaactcgccaacagtcatatgtaatagctcaagttgatgataccgttttgccaggattaggatgcgagaagcagcatgaatgtcgctcatccgatgccgcatcaccgttgtgtcagaaacgaccaagctaagcaactaaggtaccttaccgtccactatctcaggtaaccaggtactaccagctaccctacctgccgtgcctacctgctttagtattaatctttccacctccctcctcaatcttcttttccctcctctcctcttttttttttcttcctcctcttcttctccataaccattcctaacaacatcgacattctctcctaatcaccagcctcgcaaatcctcaggttagtattactactactacaatcatcaccacgatgctccgcccgacgatgcggcttctgttcgcctgcccctcctctcactcgtgcccttgacgagctaccccgccagactctcctgcgtcaccaatttttttccctatttacccctcctccctctctccctctcgtttcttcctaacaaacaaccaccaccaaaatctctttggaagctcacgactcacgcaagctcaattcgcagatacaaatctagaatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataatgcatgtgctgtgttcctcagaatgggccccagaagggcgtcgagcattgtctatgaatgcaaacaaaaatagtaaataaatagtaattctggccatgacgaatagagccaatctgctccacttgactatccttgtgactgtatcgtatgtcgaacccttgactgcccattcaaacaattgtaaaggaatatgagctacaagttatgtctcacgtttgcgtgcgagcccgtttgtacgttattttgagaaagcgttgccatcacatgctcacagtcacttggcttacgatcatgtttgcgatctttcggtaagaatacacagagtaacgattatacatccatcgctttctatgattaggtactcagacaacacatgggaaacaagataaccatcgcatgcaaggtcgattccaatcatgatctggactggggtattccatctaagccatagtacccttgcgatggctcttctggcgttctagctcggcatcactcacggcgctgagtggtgaccggcctactgcatctggctttgggcttcttttgatcttttacttctactttttctcttctacaacttgtgttaagaatgcacgaatgaacggcaaggcgctgggttccgggttaatagagcatcgtctcttttgttttctattttttttgtactctgctagggcattccttgacgggaacaaaaggtgctcgggatatcttcatgggaagcttcagtctatagatggactgagcaggcagcataacgggtcaagataccacatcaataaaagcagacttgatcatgaccttccttttcatccatcattgcaatgtcctcgtagatgtagctctagtagcaggcaatctaaatgtgaggtcaccatgatctcaagctgaagtttccacagccagtagtagacttcttaacgatgcaaatgcagccaccccgtccaatacggctcggtaggaacatttcctccaatccgtcaaccaacccatctcacgttccggctcggcagctaattaatcagcccaggccatgacaggcgacacattggatggaagttccgtcacacacatccgctaaagcatgccataccctttgctagcacaccacccacacccaaccagtcatgccgttccgtcccaccaccccctcttaatcttgggtacgtatatggatggatggatggacagacggctggaaggacggatggatggatgcatccctgagaattacccaattaggaagggcacgatgtgtggccggctgattggtccctgagcatttcattcctagtgcccttctttttccttcttcttctattttctcaaggggcagccttacgctaggtattccaatacgtaacatgttgtctcgaggtatgtcactgcagagcaggtcaaagcaagacaaggctgagtctacaggcacctgcctccattctcgtctctccgcatagtagtagtactacctatttaaccaaatcccggactacgccgaccatggacaacatgatgtgagacacatctggacagaagtgggcaatgccatttacagcctcatgcatgccacttcagcagcttgagcccttgctgcagcattatcacagcacctactgggacagtacataggtagtaggtaggaagtatgcaggtagcactacatggacatgccccctcgtggccccctatttctgcgcgcatttcacgcacgacaattcctcacacgagttggggaacggaaagattactgacaggcccttgttttatgccgactcgg

SEQ ID NO: 12 - DNA, 1615 bp, Trichoderma reesei
ctacaaccaccacatcgccaactcttccttctgcaactccaactccttcacttctcttctctgttctactcttctccacctacttcacctaccgcaatcatggccaacgcaactggcaagaaggcagtccactttggcgccggcaacatcggccgtggcttcgttgcttgcttcctccacaactcgggctatgaggtcgtttttgccgacgtcaacggagacctgattgataccatcaactctaccccctcctacaaagtcatcgaggtcggctccgagggcacctcagaaaacgtcatcaccaactaccgggccatcaactctcggactcacgaggaagacctcgttgaggagatccgcactgccgacgtcgtcacctgctccgtcggccccaacatcctcaagttcatcgctcccgtcatcgcaaagggcatcgaccgccgctccgacgacgagacgcccctgcacgtcattgcctgcgagaacgccattggcgccaccgacacgctggccagctacatcaaggatgccaagaacacggatcctgcccgtctcgagacgcaccacctgcgtgcccgctacgccaactcggctattgaccggatcgtgccggcgcaggaccccaatgccggccttgacgtgaagctggagaagttctttgagtgggttgtcgacaagaccccctttgaggacattggcatcccgcccattgagggcatccagtgggtcgacaacctggagcccttcatcgagcgcaagctgtacacggtcaacactggccacgctacggctgcctaccacggctactaccgcagaaagcgcaccgtctacgatgctctccaggacaaggagatcatggctgaggtccgtggcgccctgaccgagaccaagaacctgctcgttgccaagcacggcatcgacgaggaggcccagctcgcctacatgaacaagattatcaagcgcatcggcaacccccatttggaggatgctgtcgagcgtgtcggccgcgctcccctgaggaagctgtcgcgcaaggagcgcttcatcggccccgccgccgaggccgccgagaacgacatgcccgtcaagtacctcctcgacgccatcgagatgacgttccgcttccaggacgtcgagggtgacgacgagtccaaggagctctctaccatcatgtccgagaacacccccgaggacgttgtcgccaaggtctgcggcatcaagccgacggagaagatctaccctcgcctggtcgaggttgtcaagcgtgtgcaggctgacagccaggaggagtaacttgcgatggctcttctggcgttctagctcggcatcactcacggcgctgagtggtgaccggcctactgcatctggctttgggcttcttttgatcttttacttctactttttctcttctacaacttgtgttaagaatgcacgaatgaacggcaaggcgctgggttccgggttaatagagcatcgtctcttttgttttctattttttttgtactctgctagggcattccttgacgggaacaaaaggtgctcgggatatcttcatgggaagcttcagtctatagatggactgagcaggcagcataacgggtcaagataccacatc

SEQ ID NO: 13 - protein, 397 aa, Trichoderma reesei
MANATGKKAVHFGAGNIGRGFVACFLHNSGYEVVFADVNGDLIDTINSTPSYKVIEVGSEGTSENVITNYRAINSRTHEEDLVEEIRTADVVTCSVGPNILKFIAPVIAKGIDRRSDDETPLHVIACENAIGATDTLASYIKDAKNTDPARLETHHLRARYANSAIDRIVPAQDPNAGLDVKLEKFFEWVVDKTPFEDIGIPPIEGIQWVDNLEPFIERKLYTVNTGHATAAYHGYYRRKRTVYDALQDKEIMAEVRGALTETKNLLVAKHGIDEEAQLAYMNKIIKRIGNPHLEDAVERVGRAPLRKLSRKERFIGPAAEAAENDMPVKYLLDAIEMTFRFQDVEGDDESKELSTIMSENTPEDVVAKVCGIKPTEKIYPRLVEVVKRVQADSQEE

SEQ ID NO: 14 - DNA, 1418 bp, Trichoderma reesei
cagacaactacaaacaagatggttggcgttgacgcagagattatcgccgatgataccagaatcctgggtcaggatcctttgatccccccggctctgctcatctccgagatccccatgactcccaatgccctccagaccgtcgtcaagggccggagagacgctgcccagatcatcatgggccagaacgacaggctattagtcgtcgtcggtccctgctccatccacgaccccgaagccgcccacgagtacgccgcccgcctcaagcagctctccgagaagctcgccgacgacctctgcatcgtgatgcgcgcctacctggagaagcctcgcacgaccgtcggctggaagggcctgatcaacgatcccgatatcgactcgtccttccagatcaacaagggcctgcgcgtctcccgccggctcttcgtcgacctgacctccaacggcatgcccattgccaccgagatgctcgacaccatctccccccagttcctcgccgactgcatctccgtcggcgccattggcgcgcgaacgaccgagtcccagctgcaccgtgagctcgcttccggcctgtccttccccgtgggcttcaagaacggaaccgacggcagccttggcgtcgccattgatgccattggcgctgccgcggccaagcaccacttcatgggtgtgacgaagcagggcctggctgccattacccgcaccaagggcaacgagcacggcttcgtgatcctgagaggcggaagcaagggccccaactttgacaaggagagcgtccaggcggccaagaagatgctgacggacaagggccagaagctggccattatgattgactgttctcacggcaactctcagaagaaccacaacaaccagcccaaggttgccaaggccattgccgaccagctgcgcgagggcgagcgagccattgtcggcgtcatgatcgagtccaacatcaacgagggcaaccaaaaggtccctgctgagggtccggccggtctcaagcgcggcgttagcatcacggatgcatgcatcaactgggaggacacagtcacggtcctcgaggaccttgccgctgccgtccgcacccgacgcgaggtcaactcgggtgctcctagcgcagaggccaaggtgagcactctggaggaggactagacgtagttttaccggaaggggcgtatgtgtgtacgatgagagagaggaacgaaaagcatgagggtcggtttgttgattttgaacattacgatgcgtgagtttatgcgcgcgaatcctggcttcctccctggccagcgtaaataagaaaagttttagggagagcattcgacacggatatcgagaaattggcgcaccaaaggaacatgtatgaaggcatatattgttggctgtccactttgtcacagacggcgcacacgaaccagtttcag

SEQ ID NO: 15 - protein, 376 aa, Trichoderma reesei
MVGVDAEIIADDTRILGQDPLIPPALLISEIPMTPNALQTVVKGRRDAAQIIMGQNDRLLVVVGPCSIHDPEAAHEYAARLKQLSEKLADDLCIVMRAYLEKPRTTVGWKGLINDPDIDSSFQINKGLRVSRRLFVDLTSNGMPIATEMLDTISPQFLADCISVGAIGARTTESQLHRELASGLSFPVGFKNGTDGSLGVAIDAIGAAAAKHHFMGVTKQGLAAITRTKGNEHGFVILRGGSKGPNFDKESVQAAKKMLTDKGQKLAIMIDCSHGNSQKNHNNQPKVAKAIADQLREGERAIVGVMIESNINEGNQKVPAEGPAGLKRGVSITDACINWEDTVTVLEDLAAAVRTRREVNSGAPSAEAKVSTLEED

SEQ ID NO: 16 - DNA, 3267 bp, Trichoderma reesei
atgacaacgctgctcaacaacccgccggattcgggtcccccgccgcgccagattcgcttcgtacataaccagggccagccgccgtccaagcgacgcaggatcaatgcagcgtatgttcttctggggcttctctcgccttgcttcgtctgtcttgccgcgtcacccttggtggcaaatggctgtcctagacagataagtgcaagatggcgcgagcatcaacaccatgcggttaccagtgaagctataagtatgtagagctaacctcgtcttgcgcgcagatgcttgacctgtcgaagacgaaagacgagatgcgccggcgaacgaccgctttgcaccacatgcactaagaacgggcaccaatgcctcggttatcccgaggacaggagagatggcgaggcagcggcaaggacgggcgacgaggaaacgacgccaagggaggatgtcaaccaaggcgaggacgacgtcgcctctagaaaacccgctgcttcggggcttgcgaaccgtccgacgtcctctgggggcagcaatctgcaagctacgcatgctgctgcctctgctgcctctgcttcacggccgccgcggcgcaatatcaagcagactatcgaagtcggcgccgatgccaacccgggcgacgcagagccgcaacagtctcccgtactacatcaccaccaccagagccaccaccagaatcaccatcctcatcatcaagctcatctctcagtcacgtccgatgacttgcccagttcgcccaccctccgccgatcggcccccagccgtcgcatcccctatttccgctactttggctccacggccatcgtccccggcttcaagcaaatggtggtctctgttcgcgaccgtcgccgttcgacggcaggctcacattccggcgcctctcttcattcgacgcccagcggggcgatggccagtggctcggccattgacagcgacattgtccgcgaagacattccctcctacgaccccaacaactccgcccccgttcatcccttaatcattcagctagtcaatacgttttttgttcacgtcgggtgcaattacccctttctcaaacaagacaagtttctgcaacatgtaaaggagaaacgggtcgagccgatacttgtcgatgccgtctgcgccatcgcggcgcgcttctcggaccatcacatcctgacgggcggcaacgacaagatgccccggacagagagaggccaggtctttgcgcaacgggccaggcaggccaccgtcgacactttcccttgcccttctgtgggtgcggtccaggcttgcctgctgatggcatacgagggcttcggggcggcccaggatagcgcgctgtggatgtatctgggcttggccattcgcatggcggttgatctcggtctccaaaagtgcgtgggcgttcaatatcaaggcgaaaaggacccttggtacacccggcatcgaagtcgggccaacgacgatgatcaggacagcccagaggcgcacaaggcggatgccgtcgacgcccctagcccggaggaacagaaggaggtagagcaggagcgcatatataccttctggtccgtcttcatcctggaccggctcatctcctctggcactggacggccggtcacgatccgcgacggcgactacgagctcgcctttcccgaatccagcgtcgacctcgctaccggatggccgaacccatttcctgttctcctccagatcatccacctctacggtcaagtgtgcgatgtgctgaacaatctccacgacgcccaagaccttacccaggacaagtgggatcagctatccgagatggagcatcgactcacgagaatgtacaagcactgggacccgcggcttcagttcaacgtcaacaactttaagacgtacctcggcatgggccagggcaccaactttatcctcctccacttttggttccacgcgctcttcatcatattgcatcaaccgactcttcttacccccttttgcgaactgcggagcgagctccagctgctctcggacagccgtgagctgagcatgagcagcgcaaagaccatctgcgacattttgtcctttgccgacttgatagatccgacgagttttattggcaacccattcacgaatcagcccatttatatcgcggcatgcgcatttctcatggaatcgagcgccaacaatgcatctgagggctcgtccagggagggctcgcctcccactcaagcgagtcgctccaggcaacagacgaagcactttaacaagcagtctcggcattcgcttctcgcgtcggccgccaaccagaattaccagcgatgctacaactccctgcagcagatccaggcgtactggggcggagtaggctatatcctcaccgcgctggaccagaagtcaaagggcaagtgggactgcgagacgtataccgtggaggagtatgagagcaccaagctgccccctcctcggccgtcgattggtgagcagctgtcgcggttcgagaagcagtcgtcgcccaagattgccggctcaccgattgcttggagtctgtccggcacggccaattcgcccaactccagcctgacactcatgtatcaggctctcaacgccagcgcaggcggccacactggattccctccgcctgtgcagtccgtggcgagggctgccaatggttcgccggggagcatcgcgctggaccccattcgacacacgatgccggaagtgggaatgtttacgtcggcggttccgcagccaaacatgtctgccattcgaaactctcctcggcggccatatgctgccaattctatgaatgccgcgggggcgaactcgcggaatcagaatatcatgggcttcgaagacattccggaagagtcgccggatgcaaagggtctgatgtccaattactcaacctctggccagccctacgatggatataatgcatccccgaccagcggcaattcgggcatgggcaattctacagccgtgacgagtgcccccagtggaaacacgacgtcgaatgcgaatacccacaacaataacaacaacaacagcaccaccaccaccagcagcagcaacagcaacagcatgtatttcgcgcaaaacatggcatcatatcaagcctgggggccgatgacgggcaacatggacgccatcacgttcgacagccaggacattgacattgaggcgctggggctgcagcagccggacctgatgagcgggtggctcgagtatcttcccagtgatgtgctggggctgtttgacgagcatggcgagtcggggcagggcggccattga

SEQ ID NO: 17 - protein, 983 aa, Trichoderma reesei
MTTLLNNPPDSGPPPRQIRFVHNQGQPPSKRRRINAACLTCRRRKTRCAGERPLCTTCTKNGHQCLGYPEDRRDGEAAARTGDEETTPREDVNQGEDDVASRKPAASGLANRPTSSGGSNLQATHAAASAASASRPPRRNIKQTIENHHPHHQAHLSVTSDDLPSSPTLRRSAPSRRIPYFRYFGSTAIVPGFKQMVVSVRDRRRSTAGSHSGASLHSTPSGAMASGSAIDSDIVREDIPSYDPNNSAPVHPLIIQLVNTFFVHVGCNYPFLKQDKFLQHVKEKRVEPILVDAVCAIAARFSDHHILTGGNDKMPRTERGQVFAQRARQATVDTFPCPSVGAVQACLLMAYEGFGAAQDSALWMYLGLAIRMAVDLGLQKCVGVQYQGEKDPWYTRHRSRANDDDQDSPEAHKADAVDAPSPEEQKEVEQERIYTFWSVFILDRLISSGTGRPVTIRDGDYELAFPESSVDLATGWPNPFPVLLQIIHLYGQVCDVLNNLHDAQDLTQDKWDQLSEMEHRLTRMYKHWDPRLQFNVNNFKTYLGMGQGTNFILLHFWFHALFIILHQPTLLTPFCELRSELQLLSDSRELSMSSAKTICDILSFADLIDPTSFIGNPFTNQPIYIAACAFLMESSANNASEGSSREGSPPTQASRSRQQTKHFNKQSRHSLLASAANQNYQRCYNSLQQIQAYWGGVGYILTALDQKSKGKWDCETYTVEEYESTKLPPPRPSIGEQLSRFEKQSSPKIAGSPIAWSLSGTANSPNSSLTLMYQALNASAGGHTGFPPPVQSVARAANGSPGSIALDPIRHTMPEVGMFTSAVPQPNMSAIRNSPRRPYAANSMNAAGANSRNQNIMGFEDIPEESPDAKGLMSNYSTSGQPYDGYNASPTSGNSGMGNSTAVTSAPSGNTTNSMYFAQNMASYQAWGPMTGNMDAITFDSQDIDIEALGLQQPDLMSGWLEYLPSDVLGLFDEHGESGQGGH

SEQ ID NO: 18 - DNA, 1773 bp, Trichoderma reesei
atgtcaaccagacacatttttacgtcatgtgacggcctcgtcaacaaggccatccgaggcatcattacctacaacccttctctcagcctagacgaaagcaaccgggtcgtctatgacaccacatacgacaagtccaaagtcgtcatcataagcggaggcggctccggacatgagcctgcgtggacaggctatgtcggtcaaaacatgctcgcagcttcggttcaaggagacatctttgccagccccagcacaaagcaaatccttgcagcagtggacgctgttccgagtgacaagggcaccattctggtcatcacaaactacactggtgactgtctgcactttgggctggccaacgagaaagccaatgccaaaggccacaactgccggaccatcatctgcggcgacgatgtctctgtcggcaagaacgggagcatggttggtcgcagaggtttggctggacagatgggtgtcctcaaagtcatgggcggagctgctggtgctggaggctcgctggacgaagtttacaatcttggtgtcgcattctcgcagcagattgtgtctattgcggcaaccttggatcattgccatgttccaggacgtgctgaacatggcatgcttcaagaaggcgttgttgaactcggcaccggccctcataacgaacccggatacaagaaactatcccctgcgccatctgcgtctgatctcgtctcccagattctacaatactgtctgaatgaaaaggatccggaaagaggctacgttcacttcgcccctagtgatgaagtcatactacttctcagcaactacggcggcatatcgcacctggagatgggcgctctggttgacgaaattctcgagcagcttgcaagagactggaagattgagccggtgagggtatgcgccggcttcatcgagacgtcactcaatgctccagccttttctgtctccgtcatcaatgtgactgctgccgcaaagaattgcagttactctgtcgaagagatcaagtccttctttgatgccaggacgacgacgttttgggagtccatgactgggtcgcagacacggcggaggacgagacagcagcagattgttcagcccccggaggaagaggtcaaggcagttgatgagtcaaaagatctgactattgatcccacgctcttagagaagatgttgaggaacgcttgtgaagcgctcgtcaaggctgagccagatctcaccaagtgggataccgtcatgggtgatggagattgcggagagacgctcaggactggagccacatctttgctcacggcccttgattcgggattggcgaaatctgggtcagttgtgagagtactattggaactcgaagacatcgtggagagcaagatgggcggtactctgggcggcatattgggcatcttttttgtctccttgcgtgcagctgtcgagaagaatattggactggcccaaagcccggtcttgctctggggaacagctcttgcaacagctctggagagtctcaggaggtatacgcctgccaaggtcggagatcgtactgtcatggacacactcatcccgtttgctaccgtcatgtgtgagtcgggctttgataacggcgtcaaggcggcacaggatggagcggatgcgaccaagacgatgaagccgagactgggaagagcaacgtatgttggcgtgtcgagtgagaacaaggagttgcctcctgatcccggtgcttggggggccatggtggccattcgcggtttgcgacaaggtctggttgaagcgatctag

SEQ ID NO: 19 - protein, 590 aa, Trichoderma reesei
MSTRHIFTSCDGLVNKAIRGIITYNPSLSLDESNRVVYDTTYDKSKVVIISGGGSGHEPAWTGYVGQNMLAASVQGDIFASPSTKQILAAVDAVPSDKGTILVITNYTGDCLHFGLANEKANAKGHNCRTIICGDDVSVGKNGSMVGRRGLAGQMGVLKVMGGAAGAGGSLDEVYNLGVAFSQQIVSIAATLDHCHVPGRAEHGMLQEGVVELGTGPHNEPGYKKLSPAPSASDLVSQILQYCLNEKDPERGYVHFAPSDEVILLLSNYGGISHLEMGALVDEILEQLARDWKIEPVRVCAGFIETSLNAPAFSVSVINVTAAAKNCSYSVEEIKSFFDARTTTFWESMTGSQTRRRTRQQQIVQPPEEEVKAVDESKDLTIDPTLLEKMLRNACEALVKAEPDLTKWDTVMGDGDCGETLRTGATSLLTALDSGLAKSGSVVRVLLELEDIVESKMGGTLGGILGIFFVSLRAAVEKNIGLAQSPVLLWGTALATALESLRRYTPAKVGDRTVMDTLIPFATVMCESGFDNGVKAAQDGADATKTMKPRLGRATYVGVSSENKELPPDPGAWGAMVAIRGLRQGLVEAI

SEQ ID NO: 20 - DNA, 1326 bp, Trichoderma reesei
ttaggtgagggtgtaaccgccatcaacgcgaatatcggcaccggtcacatagcggctggcatcagacagcaggaaagccactgggcccatgagatcctgaggcagacccatcttgccctgaggaatgagggacacccatttcttcttcaggtcaggcttttcatccaggattttctcggtcctagttaaggatttgagttagccggccgaggtcacggctaccgttcaagaacagagaagagtgggttcaactcacaaagccgtcagcatgtatcccggcgagatacagttgactcggatgccagcatgcgcccactcaacggcgagcgaagccgcgagatggcggactgcggccttggaggcgttatagggcctagtccagttcaacccgttagctgtcttatgctctgctcatccatgtctcctttttttctttctttcttttttccgtcgtggaagggactcacgcttgaggctgggggacgttgacaatggcccccgacatgctgcctatcatgaccatgctaccagggctcttcctctccatgaggtgacgagcaaccgatgtagcgaagagatatgtgccgtcgacgttgacgccccagagcttgcgaactctatcaatggggtagttggtggcctcgaagttctcggtgaagccagccgaggtgaccaggttgtcaatcttgccatgttgctggatgatctcggcgatacacgcctcaacggaatcggcatctgagacgtcggcgtaatgcgccgtcactttgggaacgctagaccgggtgtcagccgtctgcctgcaaagccgtgttacgtgcagagcggagcggcagctcactcaccgttttgcgttggggttctccttgagaaaggcatccacgagcagtttcgtctgcttctcggcctcctcctctgtcgctgcgtcagctgtctggcctggggtacgagcgacccggaactggcacgcggaatctcactgttcatgtcgacgagggccaggtcggcgccagagaagaccattccctggccaatcaccagtcccagccctcttgcgccgccggtgacgacaccgacgtttccatcgagggagaagccggcaagcgtaggcttgacatactggccgccggtgccagagacgggctcgctgctcggaagctcatgctccggaggatactcgaactcgacctcggcgtcggttcgagagtggctgccgggcttatcctgccgcggcggcggcacactatggaacttcctcgcagcggccagcggcaggcgtctggcgggcgcggaggcaaaggttcggcgagccgcaagccgcagcat

SEQ ID NO: 21 - protein, 334 aa, Trichoderma reesei
MLRLAARRTFASAPARRLPLAAARKFHSVPPPRQDKPGSHSRTDAEVEFEYPPEHELPSSEPVSGTGGQYVKPTLAGFSLDGNVGVVTGGARGLGLVIGQGMVFSGADLALVDMNSEIPRASSGSLTADTRSSVPKVTAHYADVSDADSVEACIAEIIQQHGKIDNLVTSAGFTENFEATNYPIDRVRKLWGVNVDGTYLFATSVARHLMERKSPGSMVMIGSMSGAIVNVPQPQAPYNASKAAVRHLAASLAVEWAHAGIRVNCISPGYMLTALTEKILDEKPDLKKKWVSLIPQGKMGLPQDLMGPVAFLLSDASRYVTGADIRVDGGYTLT

SEQ ID NO: 22 - DNA, 640 bp, Trichoderma reesei
tcatgcctcaagatgcccaatctcttccaccttcttcgcactcgcactcgatggatcaaactcataccccagccactcagtagccaacctcctcgcaacttccagcccaacaaccctctcccccatacacaacacctgcgcattattgctctttacactgcgctcaacgctaaagctatcatgtgccgtaaccgctcgtatgcccttgaccttgttggcagaaatggcgacgcctagacctgtgccgcagatgagcagagcgcggtccgctttgccctcggcgacgaggcgggcggcggcggcggcgcgctggggatagggtgtcttgtcggagggctcgtcggatcctacgtcgatgacttggtctacgcgggcgtcgctttcaaagtcttttttgatggtgtttttgtagctgacgccggcgtcctgcggaagcgtgagtgagctcgtgtcagtgactgcccgttctgatgacctagattatgcaatcttgacgatgaagagaccgatagtcatgaagacttatgaagttgacacttgatctcgttgtgatatgagatgtgctggttctggcagccgaaacagaagcacttacatcgcagcccatggcgattctccatttcggtgccatcgtatatgt

SEQ ID NO: 23 - protein, 156 aa, Trichoderma reesei
TYTMAPKWRIAMGCDDAGVSYKNTIKKDFESDARVDQVIDVGSDEPSDKTPYPQRAAAAARLVAEGKADRALLICGTGLGVAISANKVKGIRAVTAHDSFSVERSVKSNNAQVLCMGERVVGLEVARRLATEWLGYEFDPSSASAKKVEEIGHLEA

SEQ ID NO: 24 - DNA, 726 bp, Trichoderma reesei
tcacgcttcagctacttcctgtatcgtcttgacaaactgctccggatcgtgtccgaaccgccccaggaacaatccgtccagtccgctcttcaacttctcgtagagccccggcccggcactccccccatacaaaaccctcgtcgtccccgccctcctcccaacggactccaaccctctgatcccagcaacaacatcgaggatgtgcttctctccagcaggttgactggcaccaatcgcccatacaggctcatacgccaaaatgacctctgcctcggcgggcacagcattcatgacagcatcaacctgcacccgacactcctcaaccgcgatatgcacgtcgccttgagtcttttctccaatgcacacaagcggtatcatgccattgcgacttacagcggcagccttattcgcaaccatggtgtcgtcctcgccaaagatgctcctgcgctctgcatgtccgacttccacgagcttgacgcctgcgctgcgcaacacggaggggctgacttcgccggtgaatgcgccttggtcttcccagtggcagtcttgagcgccggtccaaatggcaacggagctggacttaagctgggcgattgtggtggtcagggagacgaagtcggggatgatgaagatgtcaatgtgggagaggagctctatcgggacgtctgccagcttggataagaagctgtttgtgaagtctcttgtttgctggagagaaaagtacat

SEQ ID NO: 25 - protein, 241 aa, Trichoderma reesei
MYFSLQQTRDFTNSFLSKLADVPIELLSHIDIFIIPDFVSLTTTIAQLKSSSVAIWTGAQDCHWEDQGAFTGEVSPSVLRSAGVKLVEVGHAERRSIFGEDDTMVANKAAAVSRNGMIPLVCIGEKTQGDVHIAVEECRVQVDAVMNAVPAEAEVILAYEPVWAIGASQPAGEKHILDVVAGIRGLESVGRRAGTTRVLYGGSAGPGLYEKLKSGLDGLFLGRFGHDPEQFVKTIQEVAEA

(19) United States
(12) Patent Application Publication
(10) Pub. No.: US 2023/0357705 A1
(43) Pub. Date: Nov. 9, 2023

(54) ERYTHRITOL PRODUCING SAPROTROPH

(71) Applicant: CONZIL ESTATE GMBH, Bodman-Ludwigshafen (DE)

(72) Inventors: Robert MACH, Vienna (AT); Astrid MACH-AIGNER, Vienna (AT); Katharina REGNAT, Vienna (AT)

(21) Appl. No.: 18/012,440

(22) PCT Filed: Jun. 25, 2021
(86) PCT No.: PCT/EP2021/067473

§ 371 Date: Dec. 22, 2022

(30) Foreign Application Priority Data
Jun. 25, 2020 (EP) 20182382.0

(51) Int. Cl. C12N 1/14; C12P 7/18; C12N 9/10; C12N 9/04

(52) U.S. Cl. CPC C12N 1/145; C12P 7/18; C12N 9/1022; C12Y 202/01001; C12Y 202/01002; C12N 9/0006; C12Y 101/01017

(57) ABSTRACT

The present invention pertains to a genetically modified saprotroph for the biotechnological production of erythritol and a method for the production of erythritol using said genetically modified saprotroph.

The present invention pertains to a genetically modified saprotroph for the biotechnological production of erythritol and a method for the production of erythritol using said genetically modified saprotroph.

In the recent years people's lifestyle and the growing consumption of food products with high sugar content has resulted in a tremendous rise of blood glucose related diseases and disorders, such as diabetes mellitus type 2 (DMT2). Nowadays, a low-glycaemic nutrition and the avoidance of excessive peaks in blood glucose level is considered to reduce the risk for developing certain chronic diseases and to be beneficial for maintenance and improvement of health and for the treatment and/or prevention of a large number of blood glucose related diseases and disorders.

Erythritol is a naturally occurring four-carbon sugar alcohol gaining increasing importance in the food industry due to its specific properties and its manifold fields of application. It can be found in several fruits, such as pears, grapes and melons, mushrooms, alcoholic drinks (beer, wine, sake) and fermented food products, such as soy sauce and miso bean paste, but naturally also occurs in biofluids of humans and animals such as eye lens tissue, serum, plasma, fetal fluid and urine. Due to its small molecular weight, erythritol is easily absorbed already in the upper intestine and therefore causes less digestive distress than other sugar alcohols used in the food industry. The majority of ingested erythritol is not metabolized in the human body and is excreted unmodified into the urine without changing blood glucose and insulin levels (Regnat et al., Erythritol as sweetener wherefrom and whereto?, 2018, Applied Microbiology and Biotechnology, Vol. 102). Furthermore, erythritol is non-cariogenic, thermally stable, crystalizes well and is less hygroscopic than sucrose. Due to the negative enthalpy of dissolution, the consumption of erythritol causes a cooling sensation in the oral cavity. A 10% (w/v) solution of erythritol has 60-80% of the sweetness of sucrose.

However, in contrast to other sugar alcohols, such as sorbitol, xylitol, mannitol, lactitol, and maltitol, which are well-established as sugar alternatives for many years, so far erythritol cannot be chemically produced in a commercially worthwhile way. The production of erythritol from dialdehyde starch using a nickel catalyst at high temperatures results in unsatisfying low yields (Moon et al. Biotechnological production of erythritol and its applications, 2010, Appl. Microbiol. Biotechnol., Vol. 86).

In yeast and fungus species, erythritol is produced via the so-called pentose phosphate pathway. It is synthesized from D-erythrose-4-phosphate through dephosphorylation and subsequent reduction of erythrose. Based thereon, the suitability of osmophilic yeast, such as Aureobasidium sp., Trichosporonoides sp. and Candida magnoliae, for the biotechnological production of erythritol has been investigated in several studies (Ishizuka et al., Breeding of a mutant of Aureobasidium sp. with high erythritol production, 1989, J. Ferm. Bioeng., Vol. 68(5); U.S. Pat. Nos. 4,939,091A; 5,962,287 A; Oh et al., Increased erythritol production in fedbatch cultures of Torula sp. by controlling glucose concentration, 2001, JIM&B, Vol. 26; Koh et al., Scale-up of erythritol production by an osmophilic mutant of Candida magnoliae, 2003, Biotechnol. Lett., Vol. 25; Ryu et al., Optimization of erythritol production by Candida magnoliae in fed-batch culture, 2000, JIM&B, Vol. 25). More recent studies examined the potential of filamentous fungi to produce erythritol (Jovanovic et al., 2013). However, the yields of erythritol obtained from the different strains was unsatisfactory for industrial scale production.

Accordingly, there is still a need for a biotechnological method for the production of erythritol with increased yield. The present invention overcomes the disadvantages of the methods in the prior art by the subject-matter of the independent claims, in particular by the genetically modified saprotroph according to the present invention and the method for the production of erythritol according to the present invention.

The present invention in particular pertains to a genetically modified saprotroph comprising at least one gene encoding at least one membrane-bound alditol transporter, at least one gene encoding at least one erythrose reductase and at least one inactivated gene encoding mannitol 1-phosphate 5-dehydrogenase.

In a preferred embodiment of the present invention, the genetically modified saprotroph is a filamentous fungus. Preferably, the saprotroph is a filamentous fungus selected from the genera Hypocrea, Gibberella, Aspergillus and Penicillium. Preferably, the saprotroph is a filamentous fungus selected from Hypocrea jecorina (Trichoderma reesei), Gibberella zeae, Aspergillus niger, Aspergillus nidulans, Aspergillus oryzae and Penicillium chrysogenum.

In a particularly preferred embodiment of the present invention, the genetically modified saprotroph is Hypocrea jecorina (Trichoderma reesei). Preferably, the genetically modified saprotroph is based on the Trichoderma reesei strain QM6aΔtmus53.

In a further preferred embodiment of the present invention, the at least one gene encoding at least one membrane-bound alditol transporter is fps1, in particular codon-optimized fps1. Preferably, the at least one gene encoding at least one membrane-bound alditol transporter is fps1 from Saccharomyces cerevisiae, in particular codon-optimized fps1 from Saccharomyces cerevisiae.

In a further preferred embodiment, the at least one gene encoding at least one membrane-bound alditol transporter comprises the nucleotide sequence of SEQ ID No. 1, in particular consists of the nucleotide sequence of SEQ ID No. 1.

In a preferred embodiment of the present invention, the at least one gene encoding at least one membrane-bound alditol transporter comprises a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 1. Preferably, the at least one gene encoding at least one membrane-bound alditol transporter consists of a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 1.

Preferably, the membrane-bound alditol transporter comprises the amino acid sequence of SEQ ID No. 2, in particular consists of the amino acid sequence of SEQ ID No. 2.

Preferably, the membrane-bound alditol transporter comprises an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 2. In a further preferred embodiment, the membrane-bound alditol transporter consists of an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 2.

In another preferred embodiment of the present invention, the membrane-bound alditol transporter comprises an amino acid sequence as defined in SEQ ID No. 2, in which one, preferably two, preferably three, preferably five, preferably six, preferably seven, preferably eight, preferably nine, preferably ten, amino acids are exchanged by other naturally occurring amino acids. In a particularly preferred embodiment, the membrane-bound alditol transporter comprises an amino acid sequence as defined in SEQ ID No. 2, in which at most ten, preferably at most nine, preferably at most eight, preferably at most seven, preferably at most six, preferably at most five, preferably at most four, preferably at most three, preferably at most two, preferably at most one, amino acids are exchanged by other naturally occurring amino acids.

In a preferred embodiment of the present invention, the at least one gene encoding at least one erythrose reductase is err1, in particular codon-optimized err1. Preferably, the at least one gene encoding at least one erythrose reductase is err1, in particular codon-optimized err1, from Trichoderma reesei, Aspergillus niger or Fusarium graminearum.

In a preferred embodiment, the at least one gene encoding at least one erythrose reductase comprises the nucleotide sequence of SEQ ID No. 3, in particular consist of the nucleotide sequence of SEQ ID No. 3.

Preferably, the at least one gene encoding at least one erythrose reductase comprises a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 3. In another preferred embodiment of the present invention, the at least one gene encoding at least one gene encoding at least one erythrose reductase consists of a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 3.

Preferably, the erythrose reductase comprises the amino acid sequence of SEQ ID No. 4, in particular consists of the amino acid sequence of SEQ ID No. 4.

In a preferred embodiment, the erythrose reductase comprises an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 4. Particularly preferred, the erythrose reductase consists of an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 4.

Preferably, the erythrose reductase comprises an amino acid sequence as defined in SEQ ID No. 4, in which one, preferably two, preferably three, preferably five, preferably six, preferably seven, preferably eight, preferably nine, preferably ten, amino acids are exchanged by other naturally occurring amino acids. In a preferred embodiment of the present invention, the erythrose reductase comprises an amino acid sequence as defined in SEQ ID No. 4, in which at most ten, preferably at most nine, preferably at most eight, preferably at most seven, preferably at most six, preferably at most five, preferably at most four, preferably at most three, preferably at most two, preferably at most one, amino acids are exchanged by other naturally occurring amino acids.

In a preferred embodiment of the present invention, the at least one inactivated gene encoding mannitol 1-phosphate 5-dehydrogenase is mpdh.

Preferably, the at least one gene encoding mannitol 1-phosphate 5-dehydrogenase of the genetically modified saprotroph comprised the nucleotide sequence of SEQ ID No. 12 before inactivation, in particular consisted of the nucleotide sequence of SEQ ID No. 12 before inactivation.

Preferably, the at least one gene encoding mannitol 1-phosphate 5-dehydrogenase of the genetically modified saprotroph comprised a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 12 before inactivation. In another preferred embodiment, the at least one gene encoding mannitol 1-phosphate 5-dehydrogenase of the genetically modified saprotroph consisted of a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 12 before inactivation.

Preferably, the mannitol 1-phosphate 5-dehydrogenase encoded by the nucleotide sequence of SEQ ID No. 12 before inactivation comprises the amino acid sequence of SEQ ID No. 13, in particular consists of the amino acid sequence of SEQ ID No. 13.

In a preferred embodiment, the mannitol 1-phosphate 5-dehydrogenase encoded by the nucleotide sequence of SEQ ID No. 12 before inactivation comprises an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 13. Particularly preferred, the mannitol 1-phosphate 5-dehydrogenase encoded by the nucleotide sequence of SEQ ID No. 12 before inactivation consists of an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 13.

Preferably, the mannitol 1-phosphate 5-dehydrogenase encoded by the nucleotide sequence of SEQ ID No. 12 before inactivation comprises an amino acid sequence as defined in SEQ ID No. 13, in which one, preferably two, preferably three, preferably five, preferably six, preferably seven, preferably eight, preferably nine, preferably ten, amino acids are exchanged by other naturally occurring amino acids. In a preferred embodiment of the present invention, the mannitol 1-phosphate 5-dehydrogenase encoded by the nucleotide sequence of SEQ ID No. 12 before inactivation comprises an amino acid sequence as defined in SEQ ID No. 13, in which at most ten, preferably at most nine, preferably at most eight, preferably at most seven, preferably at most six, preferably at most five, preferably at most four, preferably at most three, preferably at most two, preferably at most one, amino acids are exchanged by other naturally occurring amino acids.

In a further preferred embodiment of the present invention, the at least one inactivated gene encoding mannitol 1-phosphate 5-dehydrogenase of the genetically modified saprotroph is deleted.

In a preferred embodiment of the present invention, the at least one inactivated gene encoding mannitol 1-phosphate 5-dehydrogenase of the genetically modified saprotroph is non-functional.

In a particularly preferred embodiment, the at least one inactivated gene encoding mannitol 1-phosphate 5-dehydrogenase of the genetically modified saprotroph is inactivated by gene knock-out, in particular gene replacement. Preferably, the at least one inactivated gene encoding mannitol 1-phosphate 5-dehydrogenase of the genetically modified saprotroph is inactivated by gene replacement using a deletion cassette.

In a preferred embodiment of the present invention, the at least one gene encoding mannitol 1-phosphate 5-dehydrogenase of the genetically modified saprotroph is inactivated, in particular made non-functional, by genome editing, in particular by meganucleases, zinc-finger nucleases (ZFNs), transcription activator-like effector nucleases (TALENs) or by the clustered regularly interspaced short palindromic repeats (CRISPR) system.

In a particularly preferred embodiment, the genetically modified saprotroph comprises at least one, preferably at least two, preferably at least three, preferably at least four, preferably at least five, further inactivated genes. Preferably, the at least one further inactivated gene is selected from the group consisting of a gene encoding phospho-2-dehydro-3-deoxyheptonate aldolase 1 (Dhaps-1), a gene encoding erythritol utilization factor (EUF), a gene encoding erythrulose kinase (EYK1), a gene encoding erythritol dehydrogenase (EYD1), a gene encoding erythritol isomerase 1 (EYI1) and a gene encoding erythritol isomerase 2 (EYI2).

In a preferred embodiment of the present invention, the at least one further inactivated gene is a gene encoding phospho-2-dehydro-3-deoxyheptonate aldolase 1 (Dhaps-1). Preferably, the at least one gene encoding phospho-2-dehydro-3-deoxyheptonate aldolase 1 (Dhaps1) of the genetically modified saprotroph comprised the nucleotide sequence of SEQ ID No. 14 before inactivation, in particular consisted of the nucleotide sequence of SEQ ID No. 14 before inactivation.

Preferably, the at least one gene encoding phospho-2-dehydro-3-deoxyheptonate aldolase 1 (Dhaps1) of the genetically modified saprotroph comprised a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 14 before inactivation. In further preferred embodiment, the at least one gene encoding phospho-2-dehydro-3-deoxyheptonate aldolase 1 (Dhaps1) of the genetically modified saprotroph consisted of a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 14 before inactivation.

Preferably, the phospho-2-dehydro-3-deoxyheptonate aldolase 1 (Dhaps1) encoded by the nucleotide sequence of SEQ ID No. 14 before inactivation comprises the amino acid sequence of SEQ ID No. 15, in particular consists of the amino acid sequence of SEQ ID No. 15.

In a preferred embodiment, the phospho-2-dehydro-3-deoxyheptonate aldolase 1 (Dhaps1) encoded by the nucleotide sequence of SEQ ID No. 14 before inactivation comprises an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 15. Particularly preferred, the phospho-2-dehydro-3-deoxyheptonate aldolase 1 (Dhaps1) encoded by the nucleotide sequence of SEQ ID No. 14 before inactivation consists of an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 15.

Preferably, the phospho-2-dehydro-3-deoxyheptonate aldolase 1 (Dhaps1) encoded by the nucleotide sequence of SEQ ID No. 14 before inactivation comprises an amino acid sequence as defined in SEQ ID No. 15, in which one, preferably two, preferably three, preferably five, preferably six, preferably seven, preferably eight, preferably nine, preferably ten, amino acids are exchanged by other naturally occurring amino acids. In a preferred embodiment of the present invention, the mannitol 1-phosphate 5-dehydrogenase encoded by the nucleotide sequence of SEQ ID No. 12 before inactivation comprises an amino acid sequence as defined in SEQ ID No. 13, in which at most ten, preferably at most nine, preferably at most eight, preferably at most seven, preferably at most six, preferably at most five, preferably at most four, preferably at most three, preferably at most two, preferably at most one, amino acids are exchanged by other naturally occurring amino acids.

In another embodiment of the present invention, the at least one further inactivated gene is a gene encoding erythritol utilization factor (EUF). Preferably, the at least one gene encoding erythritol utilization factor (Euf1) of the genetically modified saprotroph comprised the nucleotide sequence of SEQ ID No. 16 before inactivation, in particular consisted of the nucleotide sequence of SEQ ID No. 16 before inactivation.

Preferably, the at least one gene encoding erythritol utilization factor (Euf1) of the genetically modified saprotroph comprised a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 16 before inactivation. Particularly preferred, the at least one gene encoding erythritol utilization factor (Euf1) of the genetically modified saprotroph consisted of a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 16 before inactivation.

Preferably, the erythritol utilization factor (Euf1) encoded by the nucleotide sequence of SEQ ID No. 16 before inactivation comprises the amino acid sequence of SEQ ID No. 17, in particular consists of the amino acid sequence of SEQ ID No. 17.

In a preferred embodiment, the erythritol utilization factor (Euf1) encoded by the nucleotide sequence of SEQ ID No. 16 before inactivation comprises an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 17. Particularly preferred, the erythritol utilization factor (Euf1) encoded by the nucleotide sequence of SEQ ID No. 16 before inactivation consists of an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 17.

Preferably, the erythritol utilization factor (Euf1) encoded by the nucleotide sequence of SEQ ID No. 16 before inactivation comprises an amino acid sequence as defined in SEQ ID No. 17, in which one, preferably two, preferably three, preferably five, preferably six, preferably seven, preferably eight, preferably nine, preferably ten, amino acids are exchanged by other naturally occurring amino acids. In a preferred embodiment of the present invention, the erythritol utilization factor (Euf1) encoded by the nucleotide sequence of SEQ ID No. 16 before inactivation comprises an amino acid sequence as defined in SEQ ID No. 17, in which at most ten, preferably at most nine, preferably at most eight, preferably at most seven, preferably at most six, preferably at most five, preferably at most four, preferably at most three, preferably at most two, preferably at most one, amino acids are exchanged by other naturally occurring amino acids.

In a particularly preferred embodiment of the present invention, the at least one further inactivated gene is a gene encoding erythrulose kinase (EYK1). Preferably, the at least one gene encoding erythrulose kinase (Eyk1) of the genetically modified saprotroph comprised the nucleotide sequence of SEQ ID No. 18 before inactivation, in particular consisted of the nucleotide sequence of SEQ ID No. 18 before inactivation.

According to another preferred embodiment of the present invention, the at least one gene encoding erythrulose kinase (Eyk1) of the genetically modified saprotroph comprised a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 18 before inactivation. Particularly preferred, the at least one gene encoding erythrulose kinase (Eyk1) of the genetically modified saprotroph consisted of a nucleotide sequence having at least 80%, preferably at least 85%, preferably at least 90%, preferably at least 99%, sequence identity to SEQ ID No. 18 before inactivation.

Preferably, the erythrulose kinase (Eyk1) encoded by the nucleotide sequence of SEQ ID No. 18 before inactivation comprises the amino acid sequence of SEQ ID No. 19, in particular consists of the amino acid sequence of SEQ ID No. 19.

In a preferred embodiment, the erythrulose kinase (Eyk1) encoded by the nucleotide sequence of SEQ ID No. 18 before inactivation comprises an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 19. Particularly preferred, the erythrulose kinase (Eyk1) encoded by the nucleotide sequence of SEQ ID No. 18 before inactivation consists of an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 19.

Preferably, the erythrulose kinase (Eyk1) encoded by the nucleotide sequence of SEQ ID No. 18 before inactivation comprises an amino acid sequence as defined in SEQ ID No. 19, in which one, preferably two, preferably three, preferably five, preferably six, preferably seven, preferably eight, preferably nine, preferably ten, amino acids are exchanged by other naturally occurring amino acids. In a preferred embodiment of the present invention, the erythrulose kinase (Eyk1) encoded by the nucleotide sequence of SEQ ID No. 18 before inactivation comprises an amino acid sequence as defined in SEQ ID No. 19, in which at most ten, preferably at most nine, preferably at most eight, preferably at most seven, preferably at most six, preferably at most five, preferably at most four, preferably at most three, preferably at most two, preferably at most one, amino acids are exchanged by other naturally occurring amino acids.

In a preferred embodiment of the present invention, the at least one further inactivated gene is a gene encoding erythritol dehydrogenase (EYD1). Preferably, the at least one gene encoding erythritol dehydrogenase (Eyd1) of the genetically modified saprotroph comprised the nucleotide sequence of SEQ ID No. 20 before inactivation, in particular consisted of the nucleotide sequence of SEQ ID No. 20 before inactivation.

Preferably, the at least one gene encoding erythritol dehydrogenase (Eyd1) of the genetically modified saprotroph comprised a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 20 before inactivation. In another preferred embodiment of the present invention, the at least one gene encoding erythritol dehydrogenase (Eyd1) of the genetically modified saprotroph consisted of a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 20 before inactivation.

Preferably, the erythritol dehydrogenase (Eyd1) encoded by the nucleotide sequence of SEQ ID No. 20 before inactivation comprises the amino acid sequence of SEQ ID No. 21, in particular consists of the amino acid sequence of SEQ ID No. 21.

In a preferred embodiment, the erythritol dehydrogenase (Eyd1) encoded by the nucleotide sequence of SEQ ID No. 20 before inactivation comprises an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 21. Particularly preferred, the erythritol dehydrogenase (Eyd1) encoded by the nucleotide sequence of SEQ ID No. 20 before inactivation consists of an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 21.

Preferably, the erythritol dehydrogenase (Eyd1) encoded by the nucleotide sequence of SEQ ID No. 20 before inactivation comprises an amino acid sequence as defined in SEQ ID No. 21, in which one, preferably two, preferably three, preferably five, preferably six, preferably seven, preferably eight, preferably nine, preferably ten, amino acids are exchanged by other naturally occurring amino acids. In a preferred embodiment of the present invention, the erythritol dehydrogenase (Eyd1) encoded by the nucleotide sequence of SEQ ID No. 20 before inactivation comprises an amino acid sequence as defined in SEQ ID No. 21, in which at most ten, preferably at most nine, preferably at most eight, preferably at most seven, preferably at most six, preferably at most five, preferably at most four, preferably at most three, preferably at most two, preferably at most one, amino acids are exchanged by other naturally occurring amino acids.

In a preferred embodiment of the present invention, the at least one further inactivated gene is a gene encoding erythritol isomerase 1 (EYI1). Preferably, the at least one gene encoding erythritol isomerase 1 (Eyi1) of the genetically modified saprotroph comprised the nucleotide sequence of SEQ ID No. 22 before inactivation, in particular consisted of the nucleotide sequence of SEQ ID No. 22 before inactivation.

Particularly preferred, the at least one gene encoding erythritol isomerase 1 (Eyi1) of the genetically modified saprotroph comprised a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 22 before inactivation. In a further preferred embodiment of the present invention, the at least one gene encoding erythritol isomerase 1 (Eyi1) of the genetically modified saprotroph consisted of a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 22 before inactivation.

Preferably, the erythritol isomerase 1 (Eyi1) encoded by the nucleotide sequence of SEQ ID No. 22 before inactivation comprises the amino acid sequence of SEQ ID No. 23, in particular consists of the amino acid sequence of SEQ ID No. 23.

In a preferred embodiment, the erythritol isomerase 1 (Eyi1) encoded by the nucleotide sequence of SEQ ID No. 22 before inactivation comprises an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 23. Particularly preferred, the erythritol isomerase 1 (Eyi1) encoded by the nucleotide sequence of SEQ ID No. 22 before inactivation consists of an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 23.

Preferably, the erythritol isomerase 1 (Eyi1) encoded by the nucleotide sequence of SEQ ID No. 22 before inactivation comprises an amino acid sequence as defined in SEQ ID No. 23, in which one, preferably two, preferably three, preferably five, preferably six, preferably seven, preferably eight, preferably nine, preferably ten, amino acids are exchanged by other naturally occurring amino acids. In a preferred embodiment of the present invention, the erythritol isomerase 1 (Eyi1) encoded by the nucleotide sequence of SEQ ID No. 22 before inactivation comprises an amino acid sequence as defined in SEQ ID No. 23, in which at most ten, preferably at most nine, preferably at most eight, preferably at most seven, preferably at most six, preferably at most five, preferably at most four, preferably at most three, preferably at most two, preferably at most one, amino acids are exchanged by other naturally occurring amino acids.

In another preferred embodiment of the present invention, the at least one further inactivated gene is a gene encoding erythritol isomerase 2 (EYI2). Preferably, the at least one gene encoding erythritol isomerase 2 (Eyi2) of the genetically modified saprotroph comprised the nucleotide sequence of SEQ ID No. 24 before inactivation, in particular consisted of the nucleotide sequence of SEQ ID No. 24 before inactivation.

Preferably, the at least one gene encoding erythritol isomerase 2 (Eyi2) of the genetically modified saprotroph comprised a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 24 before inactivation. In a preferred embodiment of the present invention, the at least one gene encoding erythritol isomerase 2 (Eyi2) of the genetically modified saprotroph consisted of a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 24 before inactivation.

Preferably, the erythritol isomerase 2 (Eyi2) encoded by the nucleotide sequence of SEQ ID No. 24 before inactivation comprises the amino acid sequence of SEQ ID No. 25, in particular consists of the amino acid sequence of SEQ ID No. 25.

In a preferred embodiment, the erythritol isomerase 2 (Eyi2) encoded by the nucleotide sequence of SEQ ID No. 24 before inactivation comprises an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 25. Particularly preferred, the erythritol isomerase 2 (Eyi2) encoded by the nucleotide sequence of SEQ ID No. 24 before inactivation consists of an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 25.

Preferably, the erythritol isomerase 2 (Eyi2) encoded by the nucleotide sequence of SEQ ID No. 24 before inactivation comprises an amino acid sequence as defined in SEQ ID No. 25, in which one, preferably two, preferably three, preferably five, preferably six, preferably seven, preferably eight, preferably nine, preferably ten, amino acids are exchanged by other naturally occurring amino acids. In a preferred embodiment of the present invention, the erythritol isomerase 2 (Eyi2) encoded by the nucleotide sequence of SEQ ID No. 24 before inactivation comprises an amino acid sequence as defined in SEQ ID No. 25, in which at most ten, preferably at most nine, preferably at most eight, preferably at most seven, preferably at most six, preferably at most five, preferably at most four, preferably at most three, preferably at most two, preferably at most one, amino acids are exchanged by other naturally occurring amino acids.

In a further preferred embodiment of the present invention, the at least one further inactivated gene selected from the group consisting of a gene encoding phospho-2-dehydro-3-deoxyheptonate aldolase 1 (Dhaps-1), a gene encoding erythritol utilization factor (EUF), a gene encoding erythrulose kinase (EYK1), a gene encoding erythritol dehydrogenase (EYD1), a gene encoding erythritol isomerase (EYI1) and a gene encoding erythritol isomerase (EYI2) of the genetically modified saprotroph is deleted.

Preferably, the at least one further inactivated gene selected from the group consisting of a gene encoding phospho-2-dehydro-3-deoxyheptonate aldolase 1 (Dhaps-1), a gene encoding erythritol utilization factor (EUF), a gene encoding erythrulose kinase (EYK1), a gene encoding erythritol dehydrogenase (EYD1), a gene encoding erythritol isomerase (EYI1) and a gene encoding erythritol isomerase (EYI2) of the genetically modified saprotroph is non-functional.

In a particularly preferred embodiment, the at least one further inactivated gene selected from the group consisting of a gene encoding phospho-2-dehydro-3-deoxyheptonate aldolase 1 (Dhaps-1), a gene encoding erythritol utilization factor (EUF), a gene encoding erythrulose kinase (EYK1), a gene encoding erythritol dehydrogenase (EYD1), a gene encoding erythritol isomerase (EYI1) and a gene encoding erythritol isomerase (EYI2) of the genetically modified saprotroph is inactivated by gene knock-out, in particular gene replacement. Preferably, the at least one further inactivated gene selected from the group consisting of a gene encoding phospho-2-dehydro-3-deoxyheptonate aldolase 1 (Dhaps-1), a gene encoding erythritol utilization factor (EUF), a gene encoding erythrulose kinase (EYK1), a gene encoding erythritol dehydrogenase (EYD1), a gene encoding erythritol isomerase (EYI1) and a gene encoding erythritol isomerase (EYI2) of the genetically modified saprotroph is inactivated by gene replacement using a deletion cassette.

In a preferred embodiment of the present invention, the at least one further inactivated gene selected from the group consisting of a gene encoding phospho-2-dehydro-3-deoxyheptonate aldolase 1 (Dhaps-1), a gene encoding erythritol utilization factor (EUF), a gene encoding erythrulose kinase (EYK1), a gene encoding erythritol dehydrogenase (EYD1), a gene encoding erythritol isomerase (EYI1) and a gene encoding erythritol isomerase (EYI2) of the genetically modified saprotroph is inactivated, in particular made non-functional, by genome editing, in particular by meganucleases, zinc-finger nucleases (ZFNs), transcription activator-like effector nucleases (TALENs) or by the clustered regularly interspaced short palindromic repeats (CRISPR) system.

In a further preferred embodiment of the present invention, the genetically modified saprotroph further comprises at least one gene encoding at least one transketolase. Preferably, the at least one gene encoding at least one transketolase is tkl1, in particular codon-optimized tkl1. Preferably, the genetically modified saprotroph further comprises at least one gene encoding tkl1 from T. reesei, in particular codon-optimized tkl1 from T. reesei. In a preferred embodiment, the at least one gene encoding at least one transketolase comprises the nucleotide sequence of SEQ ID No. 5, in particular consists of the nucleotide sequence of SEQ ID No. 5.

In a further preferred embodiment of the present invention, the at least one gene encoding at least one transketolase comprises a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 5. Preferably, the at least one gene encoding at least one transketolase consists of a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 5.

Preferably, the at least one transketolase comprises the amino acid sequence of SEQ ID No. 6, in particular consists of the amino acid sequence of SEQ ID No. 6.

In another preferred embodiment of the present invention, the at least one transketolase comprises an amino acid sequence having at least 80%, preferably at least 85%, preferably at least 90%, preferably at least 99%, sequence identity to SEQ ID No. 6. Particularly preferred, the at least one transketolase consists of an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 6.

In a further preferred embodiment of the present invention, the at least one transketolase comprises an amino acid sequence as defined in SEQ ID No. 6, in which one, preferably two, preferably three, preferably five, preferably six, preferably seven, preferably eight, preferably nine, preferably ten, amino acids are exchanged by other naturally occurring amino acids. In a particularly preferred embodiment, the at least one transketolase comprises an amino acid sequence as defined in SEQ

ID No. 6, in which at most ten, preferably at most nine, preferably at most eight, preferably at most seven, preferably at most six, preferably at most five, preferably at most four, preferably at most three, preferably at most two, preferably at most one, amino acids are exchanged by other naturally occurring amino acids. In a preferred embodiment of the present invention, the genetically modified saprotroph further comprises at least one gene encoding at least one transaldolase.

Preferably, the at least one gene encoding at least one transaldolase is tal1, in particular codon-optimized tal1. Preferably, the genetically modified saprotroph further comprises at least one gene encoding tal1 from T. reesei, in particular codon-optimized tal1 from T. reesei. In a preferred embodiment, the at least one gene encoding at least one transaldolase comprises the nucleotide sequence of SEQ ID No. 7, in particular consist of the nucleotide sequence of SEQ ID No. 7.

In another preferred embodiment, the at least one gene encoding at least one transaldolase comprises a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 7. Preferably, the at least one gene encoding at least one transaldolase consists of a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 7.

Preferably, the at least one transaldolase comprises the amino acid sequence of SEQ ID No. 8, in particular consists of the amino acid sequence of SEQ ID No. 8.

Particularly preferred, the at least one transaldolase comprises an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 8. Preferably, the at least one transaldolase consists of an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 8.

In another preferred embodiment of the present invention, the at least one transaldolase comprises an amino acid sequence as defined in SEQ ID No. 8, in which one, preferably two, preferably three, preferably five, preferably six, preferably seven, preferably eight, preferably nine, preferably ten, amino acids are exchanged by other naturally occurring amino acids. In a particularly preferred embodiment, the at least one transaldolase comprises an amino acid sequence as defined in SEQ ID No. 8, in which at most ten, preferably at most nine, preferably at most eight, preferably at most seven, preferably at most six, preferably at most five, preferably at most four, preferably at most three, preferably at most two, preferably at most one, amino acids are exchanged by other naturally occurring amino acids.

In a further preferred embodiment of the present invention, the genetically modified saprotroph further comprises at least one gene encoding at least one erythritol utilization factor (EUF). Preferably, the at least one gene encoding at least one erythritol utilization factor, is euf1, in particular codon-optimized euf1. Preferably, the genetically modified saprotroph further comprises at least one gene encoding euf1 from T. reesei, in particular codon-optimized euf1 from T. reesei. In a preferred embodiment, the at least one gene encoding at least one erythritol utilization factor (EUF) comprises the nucleotide sequence of SEQ ID No. 16, in particular consist of the nucleotide sequence of SEQ ID No. 16.

In a preferred embodiment, the at least one gene encoding at least one erythritol utilization factor comprises a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 16. Preferably, the at least one gene encoding at least one erythritol utilization factor consists of a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 16.

Preferably, the at least one erythritol utilization factor (EUF) comprises the amino acid sequence of SEQ ID No. 17, in particular consists of the amino acid sequence of SEQ ID No. 17.

In a further preferred embodiment, the at least one erythritol utilization factor comprises an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 17. Preferably, the at least one erythritol utilization factor consists of an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 17.

In a further preferred embodiment of the present invention, the at least one erythritol utilization factor comprises an amino acid sequence as defined in SEQ ID No. 17, in which one, preferably two, preferably three, preferably five, preferably six, preferably seven, preferably eight, preferably nine, preferably ten, amino acids are exchanged by other naturally occurring amino acids. In a particularly preferred embodiment, the at least one erythritol utilization factor comprises an amino acid sequence as defined in SEQ ID No. 17, in which at most ten, preferably at most nine, preferably at most eight, preferably at most seven, preferably at most six, preferably at most five, preferably at most four, preferably at most three, preferably at most two, preferably at most one, amino acids are exchanged by other naturally occurring amino acids.

In a preferred embodiment of the present invention, the at least one gene encoding at least one membrane-bound alditol transporter, the at least one gene encoding at least one erythrose reductase, the at least one gene encoding at least one transketolase, the at least one gene encoding at least one transaldolase and/or the at least one gene encoding at least one erythritol utilization factor (EUF) of the genetically modified saprotroph is an exogenous polynucleotide sequence.

Preferably, the at least one gene encoding at least one membrane-bound alditol transporter, the at least one gene encoding at least one erythrose reductase, the at least one gene encoding at least one transketolase, the at least one gene encoding at least one transaldolase and/or the at least one gene encoding at least one erythritol utilization factor (EUF) of the genetically modified saprotroph is an endogenous polynucleotide sequence.

In a further preferred embodiment, the genetically modified saprotroph according to the present invention comprises at least one exogenous gene encoding at least one membrane-bound alditol transporter. Preferably, genetically modified saprotroph according to the present invention comprises at least one endogenous gene encoding at least one membrane-bound alditol transporter.

Preferably, the genetically modified saprotroph according to the present invention comprises at least one exogenous gene encoding at least one erythrose reductase. In another preferred embodiment, the genetically modified saprotroph according to the present invention comprises at least one endogenous gene encoding at least one erythrose reductase.

Further preferred, the genetically modified saprotroph according to the present invention comprises at least one exogenous gene encoding at least one transketolase. In a preferred embodiment of the present invention, the genetically modified saprotroph according to the present invention comprises at least one endogenous gene encoding at least one transketolase.

Preferably, the genetically modified saprotroph according to the present invention comprises at least one exogenous gene encoding at least one transaldolase. Preferably, the genetically modified saprotroph according to the present invention comprises at least one endogenous gene encoding at least one transaldolase.

Preferably, the genetically modified saprotroph according to the present invention comprises at least one exogenous gene encoding at least one erythritol utilization factor (EUF). Preferably, the genetically modified saprotroph according to the present invention comprises at least one endogenous gene encoding at least one erythritol utilization factor (EUF).

In a preferred embodiment of the present invention, the at least one gene encoding at least one membrane-bound alditol transporter, the at least one gene encoding at least one erythrose reductase, the at least one gene encoding at least one transketolase, the at least one gene encoding at least one transaldolase and/or the at least one gene encoding at least one erythritol utilization factor (EUF) is stably or transiently introduced into the genome of the genetically modified saprotroph.

In a particularly preferred embodiment of the present invention, the at least one gene encoding at least one membrane-bound alditol transporter, the at least one gene encoding at least one erythrose reductase, the at least one gene encoding at least one transketolase, the at least one gene encoding at least one transaldolase and/or the at least one gene encoding at least one erythritol utilization factor (EUF), is overexpressed.

Preferably, the at least one gene encoding at least one membrane-bound alditol transporter, in particular the at least one exogenous or endogenous gene encoding at least one membrane-bound alditol transporter, is overexpressed. In a further preferred embodiment, the at least one gene encoding at least one erythrose reductase, in particular the at least one exogenous or endogenous gene encoding at least one erythrose reductase, is overexpressed. Preferably, the at least one gene encoding at least one transketolase, in particular the at least one exogenous or endogenous gene encoding at least one transketolase, is overexpressed. Further preferred, the at least one gene encoding at least one transaldolase, in particular the at least one exogenous or endogenous gene encoding at least one transaldolase, is overexpressed. Preferably, the at least one gene encoding at least one erythritol utilization factor (EUF), in particular the at least one exogenous or endogenous gene encoding at least one erythritol utilization factor (EUF), is overexpressed.

In a further preferred embodiment of the present invention, the at least one gene encoding at least one membrane-bound alditol transporter, the at least one gene encoding at least one erythrose reductase, the at least one gene encoding at least one transketolase, the at least one gene encoding at least one transaldolase and/or the at least one gene encoding at least one erythritol utilization factor (EUF) is under the control of a constitutive or inducible promoter. In a preferred embodiment, the constitutive or inducible promoter is a naturally occurring promoter. In a further preferred embodiment, the constitutive or inducible promoter is a synthetic promoter. Preferably, the constitutive promoter is selected from pki, tef, gpd. The inducible promoter is preferably selected from bga1, bxl1, cbh1, cbh2, xyn1, xyn2. Preferably, the at least one gene encoding at least one membrane-bound alditol transporter is under the control of a promoter selected from pki, tef, gpd, preferably under control of a pki promoter. Preferably, the at least one gene encoding at least one erythrose reductase is under the control of a promoter selected from pki, tef, gpd, preferably under control of a tef promoter. Preferably, the at least one gene encoding at least one transketolase is under the control of a promoter selected from pki, tef, gpd, preferably under control of a tef promoter. Preferably, the at least one gene encoding at least one transaldolase is under the control of a promoter selected from pki, tef, gpd, preferably under control of a tef promoter. Preferably, the at least one gene encoding at least one erythritol utilization factor (EUF) is under the control of a promoter selected from pki, tef, gpd, preferably under control of a tef promoter.

In a further preferred embodiment of the present invention, the at least one gene encoding at least one membrane-bound alditol transporter, the at least one gene encoding at least one erythrose reductase, the at least one gene encoding at least one transketolase, the at least one gene encoding at least one transaldolase and/or the at least one gene encoding at least one erythritol utilization factor (EUF) is present on a plasmid. Preferably, each of the at least one gene encoding at least one membrane-bound alditol transporter, the at least one gene encoding at least one erythrose reductase, the at least one gene encoding at least one transketolase, the at least one gene encoding at least one transaldolase and/or the at least one gene encoding at least one erythritol utilization factor (EUF) is present on a separate plasmid.

In a particularly preferred embodiment of the present invention, the genetically modified saprotroph is the Trichoderma reesei strain deposited at the Westerdijk Fungal Biodiversity Institute under CBS number 146708.

The present invention further pertains to a method for the production of erythritol, comprising the steps:
a) providing at least one genetically modified saprotroph according to the present invention,
b) culturing the at least one genetically modified saprotroph provided in step a) in the presence of a culture medium, so as to obtain erythritol in the culture medium,
c) recovering erythritol from the culture medium.

In a preferred embodiment of the present invention, the culture medium comprises lignocellulosic biomass, in particular straw, and/or at least one residue of dairy product production, in particular whey. Particularly preferred, the culture medium comprises lignocellulosic biomass, in particular straw, preferably hydrolysed straw. According to this particular embodiment, the genetically modified saprotroph is able to grow on lignocellulosic biomass as carbon source, in particular on lignocellulosic biomass as the sole carbon source. In a further preferred embodiment of the present invention, the culture medium comprises at least one residue of dairy product production. Particularly preferred, the culture medium comprises whey. In this way, the method according to the present invention advantageously utilizes inexpensive, abundant and renewable substrates as educts for the production of erythritol.

In a preferred embodiment of the present invention, the culture medium comprises ammonium as a nitrogen source. In a further preferred embodiment of the present invention, the culture medium comprises nitrate as a nitrogen source.

In a further preferred embodiment of the present invention, the culture medium comprises glycerol.

In a preferred embodiment of the present invention, the at least one genetically modified saprotroph provided in step a) is cultured in step b) at a temperature of 25 to 35° C., preferably 26 to 34° C., preferably 27 to 33° C., preferably 28 to 32° C., preferably 29 to 31° C., preferably 30° C.

Preferably, the pH of the culture medium in step b) is within the range of 3 to 8, preferably 3 to 7, preferably 3.5 to 6.5, preferably 3.5 to 6, preferably 4 to 5.5, preferably 4 to 5.

In a particularly preferred embodiment of the present invention, step b) is conducted until a concentration of erythritol in the culture medium of at least 1 g/L, preferably at least 1.5 g/L, preferably at least 2 g/L, preferably at least 2.5 g/L, preferably at least 3 g/L, is reached.

In a preferred embodiment of the present invention, in step b) at least one substrate that causes osmotic stress is added to the culture medium. Preferably, the at least one substrate that causes osmotic stress is added to the culture medium after 10 hours of cultivation, preferably 12 hours of cultivation, preferably 14 hours of cultivation, preferably 16 hours of cultivation.

In a further preferred embodiment of the present invention, the substrate that causes osmotic stress is selected from glycerol and sodium chloride or a combination thereof. Preferably, the substrate that causes osmotic stress is glycerol. Preferably, the substrate that causes osmotic stress is sodium chloride.

In a preferred embodiment of the present invention, in step c) erythritol is recovered by crystallisation. Particularly preferred, the recovery of erythritol from the culture medium in step c) comprises the steps:
i) removal of biomass, preferably removal of biomass by filtration,
ii) decolorisation of the culture medium, preferably decolorisation of the culture medium with active carbon,
iii) desalting the culture medium, preferably desalting and decolorizing the culture medium with at least one ion exchange resin, and
iv) concentrating and crystallising the erythritol.

The present invention also pertains to the use of a genetically modified saprotroph according to the present invention for the production of erythritol.

The term “genetically modified” and its grammatical equivalents as used herein refer to one or more alterations of a nucleic acid, e.g. the nucleic acid within the genome of an organism, in particular within the genome of a saprotroph. A “genetically modified” saprotroph can refer to a saprotroph with an added, deleted and/or altered, in particular inactivated, gene.

In the context of the present invention, the term “erythrose reductase” pertains to any enzyme that catalyses reversibly the reduction of an aldose to an alditol, wherein the enzyme has predominant specific activity for the reduction of erythrose to erythritol. Thus, an “erythrose reductase” according to the present invention exhibits a high specificity and affinity for the substrate erythrose. Particularly, the “erythrose reductase” has a higher specificity for erythrose than for any other substrate, such as glycerol.

The term “membrane-bound alditol transporter” as used in the context of the present invention designates a membrane protein suitable to reversibly transport alditols across the outer membrane, in particular to transport intracellularly produced alditols to the culture medium. According to the present invention, a “membrane-bound alditol transporter” is suitable to transport intracellularly produced erythritol across the membrane to the culture medium.

In the context of the present invention, the term “transketolase” refers to an enzyme of the pentose phosphate pathway catalysing the thiamine-dependent reversible transfer of a C-2 unit from a ketose donor to an acceptor aldopentose. In particular, the “transketolase” catalyses the transfer of a C-2 unit from xylulose-5-phosphate to ribose-5-phosphate forming seduheptulose-7-phosphate and glyceraldehyde-3-phosphate.

The term “transaldolase” designates an enzyme of the non-oxidative branch of the pentose phosphate pathway catalysing the reversible transfer of a C-3 unit from a ketose donor to an acceptor aldose. In particular, the “transketolase” catalyses the transfer of a C-3 unit from sedoheptulose-7-phosphate to glyceraldehyde-3-phosphate forming erythrose-4-phosphate and fructose-6-phosphate.

In the context of the present invention an “inactivated” gene is a gene which can temporarily or permanently not be transcribed or can be transcribed but the transcript is not accessible for gene translation. According to the present invention, the inactivation of a gene includes but is not limited to inactivation by partial or complete gene deletion, partial or complete gene replacement, gene repression, gene insertion, and gene mutation. “Inactivation” in the context of the present invention further includes any post-transcriptional gene regulation, in particular by RNAi or siRNA, resulting in inhibition of translation of the transcript into the gene product.

In the context of the present invention a “non-functional” gene is a gene that is present in the genome of an organism or a plasmid in an organism which gene is either not transcribed or is transcribed but the transcript is not translated to the gene product.

In the context of the present invention a “deleted” gene is a gene that has previously been present in the genome of an organism or a plasmid in an organism but has been completely removed from the genome of the organism or the plasmid in the organism.

In the context of the present invention the verb “to overexpress” refers to the artificial expression of a gene in increased quantity. This includes the inducible overexpression of a gene which can be regulated using an inducible promoter and the constant overexpression of a gene using a constitutive promoter.

In the context of the present invention, the term “a” is meant to include the meaning of “one” or “one or more”.

In the context of the present invention, the term “comprising” preferably has the meaning of “containing” or “including”, meaning including the specifically identified elements without excluding the presence of further elements. However, in a preferred embodiment, the term “comprising” is also understood to have the meaning of “consisting essentially of” and in a further preferred embodiment the meaning of “consisting of”.

The terms “and/or” is used herein to express the disclosure of any combination of individual elements connected within a listing. Solely for illustrative purposes, the expression “A, B, and/or C” can mean A individually, B individually, C individually, (A and B), (B and C), (A and C), and (A, B and C).

Further preferred embodiments of the invention are subject of the subclaims.

The invention is further described by way of the following example and the accompanying figures.

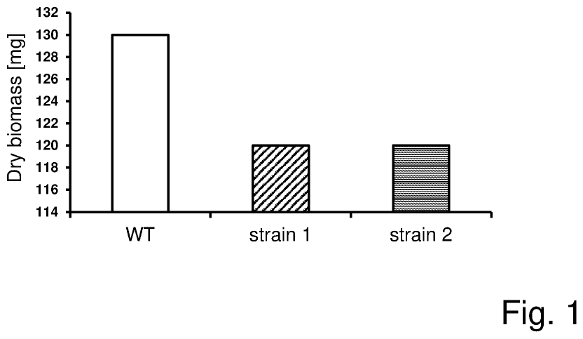

FIG. 1 shows a comparison dry biomass of a T. reesei wild-type strain (WT), of a T. reesei strain (strain 1) overexpressing the alditol transporter gene fps1 and comprising an inactivated gene encoding mannitol 1-phosphate 5-dehydrogenase (mpdh) and of a T. reesei strain (strain 2) overexpressing the alditol transporter gene fps1 and the gene err1 encoding erythrose reductase as well as comprising an inactivated gene encoding mannitol 1-phosphate 5-dehydrogenase (mpdh) after 40 hours of cultivation.

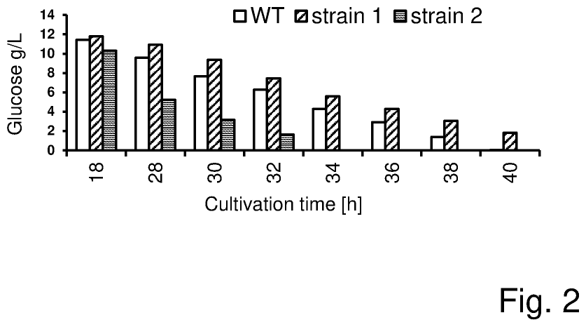

FIG. 2 shows the concentration of glucose in the culture medium after 18, 28, 30, 32, 34, 36, 38 and 40 hours of cultivation of the wild-type strain (WT) and strains 1 and 2.

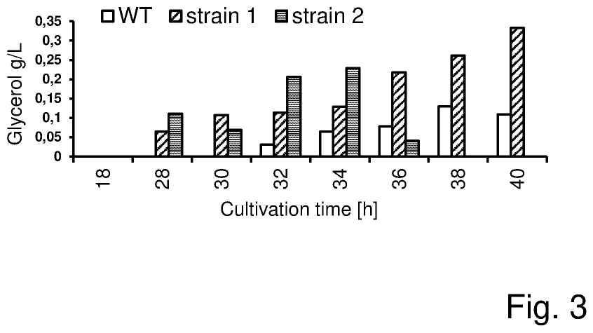

FIG. 3 shows the concentration of glycerol in the culture medium after 18, 28, 30, 32, 34, 36, 38 and 40 hours of cultivation of the wild-type strain (WT) and strains 1 and 2.

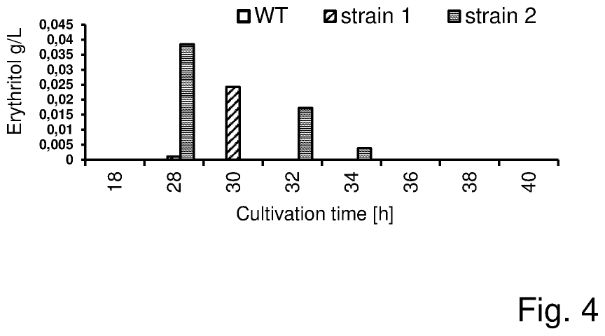

FIG. 4 shows the concentration of erythritol in the culture medium after 18, 28, 30, 32, 34, 36, 38 and 40 hours of cultivation of the wild-type strain (WT) and strains 1 and 2.

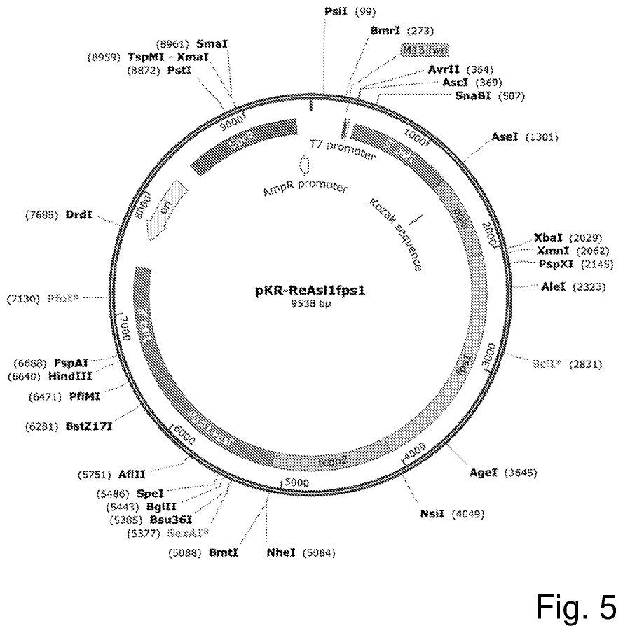

FIG. 5 shows a plasmid map of pKR_ReAsl1fps1 (see also SEQ ID No. 9).

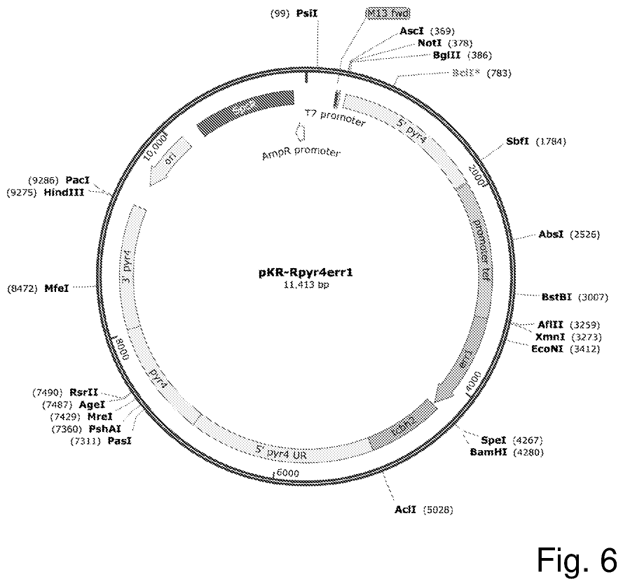

FIG. 6 shows a plasmid map of pKR_Rpyr4err1 (see also SEQ ID No 0.10).

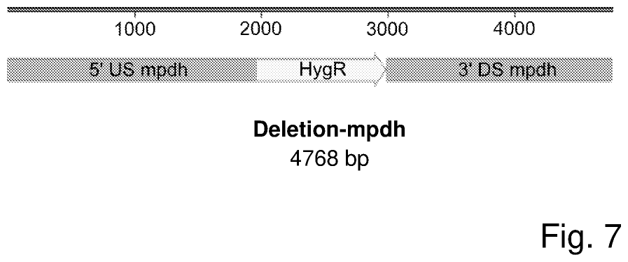

FIG. 7 shows a deletion cassette for deletion of mpdh (see also SEQ ID No. 11).

### EXAMPLE

1.1 Comparison of T. reesei Strains

For the characterization of the phenotype the following strains were used:
the wild type strain QM6aΔtmus53, short-termed “WT”,
QM6aΔtmus53 fps1(Reasl1)Δmpdh(hygR), short-termed “strain 1”, and
QM6aΔtmus53 fps1(Reasl1)OEerr1(Repyr4)Δmpdh(hygR), short-termed “strain 2”. This strain has been deposited on May 25, 2020 in the Restricted CBS collection of the Westerdijk Fungal Biodiversity Institute, Utrecht, The Netherlands by the depositor Technische Universität Wien (Gumpendorfer Straβe 1a, 1060 Vienna, Austria) under the stipulations of the Budapest Treaty on the International Recognition of the Deposit of Microorganisms for the Purpose of Patent Procedure. The corresponding viability certificate has been emitted and the strain has been given accession number CBS 146708.

1.2 Materials and Methods

1.2.1 Generation of T. reesei Strains

The parental T. reesei strain used in the experiments is auxotroph for uridine (lack of functional pyr4), arginine (lack of functional asl1), histidine (lack of functional 80820 HisG) and is further resistant to hygromycin (hygR). The first transformation with the plasmid pKR_Reasl1fps1 (see FIG. 5) to restore the asl1 locus not only introduces the fps1 gene into the genome, but also makes the strain hygromycin sensible again. The err1 gene encoding erythrose reductase was introduced using the plasmid pKR_Repyr4err1 (see FIG. 6). The deletion of mpdh was done by transformation of a deletion cassette (see FIG. 7) to regain hygromycin resistance by homologous recombination. All transformations are performed as protoplast transformations. Typically, 30 to 50 μg of digested plasmid DNA (in 15 μl sterile double-distilled water) were used for transformation of 10⁷ protoplasts (in 200 μl). For selection for resistance against hygromycin B, 100 μl to 2 ml of the transformation reaction mixture was added to 10 ml melted, 50° C. warm malt extract (MEX) agar containing 1.2 M sorbitol. This mixture was poured into sterile petri dishes and incubated at 30° C. for 5 h after solidification. Subsequently, 10 ml melted, 50° C. MEX agar containing 1.2 M sorbitol and a double concentration of hygromycin B was poured on top of the protoplast-containing layer. Plates were incubated at 30° C. for 2 to 5 days until colonies were visible. For selection for prototrophy, 100 μl to 2 ml of the transformation reaction mixture was added to 20 ml melted, 50° C. warm minimal medium agar containing 1.2 M sorbitol and, in the case of asl1 back transformation, additionally 0.25 mM L-arginine. This mixture was poured into sterile petri dishes and after solidification was incubated at 30° C. for 3 to 7 days until colonies were visible.

1.2.2 Cultivation of T. reesei and Induction of Erythritol Synthesis

In the experiment ammonium was used as nitrogen source and glucose as carbon source under osmotic stress. The total duration of cultivation was 40 hours. After 10 hours of cultivation for biomass production, sodium chloride was added to the medium in order to initiate osmotic stress. Culture broth samples have been taken at the time of this addition and next after 28 hours of total cultivation time and then every two hours until end of the cultivation. Samples were centrifuged to separate the mycelium from the supernatant.

Cultivation was performed with the wild-type strain (WT), QM6a_fm (strain 1) and QM6a_fem (strain 2) in 100 ml shake flasks containing 25 ml cultivation medium ((NH₄)₂SO₄ 2.80 g/l, KH₂PO₄ 4.00 g/l, MgSO₄x7H₂O 1.0 g/l, NaCl 0.5 g/l, peptone from Casein 0.1 g/l, Tween®80 0.5 g/l), supplemented with 1.5 ml/l trace element solution (FeSO₄x7H₂O 0.90 mM, MnSO₄xH₂O 0.50 mM, ZnSO₄x7H₂O 0.24 mM, CaCl₂x7H₂ 0.68 mM). For inoculation 10⁹ conidiospores per liter were used. To all flasks 0.3 ml of 0.2 M histidine and 0.25 ml of 0.2 M uridine was added. After 10 hours 0.5 ml of 5 M NaCl was added. The agitation rate was 180 rpm, the temperature was 30° C.

After the cultivation the whole cultivation broth was filtered through miracloth, the mycelia were pressed dry with Whatman filter paper and dried at 80° C. overnight.

Glucose, glycerol, mannitol and erythritol concentrations were quantified by high-performance liquid chromatography (HPLC) (Shimadzu Prominence Series equipped with a RID-20A refractive-index detector). Culture supernatants were filtered through 0.20 μm membranes and measured by HPLC using a Rezex RCM-Monosaccharide column (300 mm×7.8 mm; Phenomenex, Torrance, CA, USA). The mobile phase was 30% acetonitrile (HPLC grade). For sample analysis, the column was eluted at 60° C. with 30% acetonitrile (HPLC grade) at a flow rate of 0.6 ml/min.

1.3 Results

The biomass production at the end of the cultivation is nearly the same for all strains. WT produces slightly more biomass (FIG. 1). The glucose consumption is faster in strain 2 compared to WT and strain 1, which is deduced from concentration measurements in the respective supernatants (FIG. 2).

The glycerol concentration in the supernatant of strain 1 is always higher than in the WT due to the deletion of mannitol 1-phosphate 5-dehydrogenase (FIG. 3) since this deletion results in elimination of the production of mannitol and the strains produce more of the osmolyte glycerol. Whereas strain 1 shows a constant increase in glycerol production, strain 2 bearing the overexpressed err1 gene shows a fluctuating accumulation of glycerol over time (FIG. 3). Erythritol is only detectable in the supernatants of strains bearing the transporter FPS1 of Saccharomyces cerevisiae (FIG. 4). Investigations on the consumption of erythritol demonstrated that all strains, including WT, can grow on erythritol as sole carbon source (data not shown). The presence of erythritol in the supernatant seems to coincide with the secretion of glycerol and it is higher for strain 2 (FIG. 3 and FIG. 4).

1. A genetically modified saprotroph comprising at least one gene encoding at least one membrane-bound alditol transporter, at least one gene encoding at least one erythrose reductase and at least one inactivated gene encoding mannitol 1-phosphate 5-dehydrogenase.

2. The genetically modified saprotroph according to claim 1, wherein the saprotroph is a filamentous fungus selected from the genera Hypocrea, Gibberella, Aspergillus and Penicillium.

3. The genetically modified saprotroph according to claim 1, wherein the saprotroph is Hypocrea jecorina (Trichoderma reesei).

4. The genetically modified saprotroph according to claim 1, wherein the at least one gene encoding at least one membrane-bound alditol transporter is fps1.

5. The genetically modified saprotroph according to claim 1, wherein the at least one gene encoding at least one erythrose reductase is err1.

6. The genetically modified saprotroph according to claim 1, wherein the at least one inactivated gene encoding mannitol 1-phosphate 5-dehydrogenase is mpdh.

7. The genetically modified saprotroph according to claim 1, wherein the saprotroph further comprises at least one gene encoding at least one transketolase, in particular tkl1.

8. The genetically modified saprotroph according to claim 1, wherein the saprotroph further comprises at least one gene encoding at least one transaldolase, in particular tal1.

9. The genetically modified saprotroph according to claim 1, wherein the saprotroph further comprises at least one inactivated gene encoding phospho-2-dehydro-3-deoxyheptonate aldolase 1 (Dhaps-1).

10. The genetically modified saprotroph according to claim 1, wherein the at least one gene encoding at least one membrane-bound alditol transporter, the at least one gene encoding at least one erythrose reductase, the at least one gene encoding at least one transketolase and/or the at least one gene encoding at least one transaldolase is overexpressed.

11. A method for the production of erythritol, comprising the steps:
a) providing at least one genetically modified saprotroph according to claim 1,
b) culturing the at least one genetically modified saprotroph provided in step a) in the presence a culture medium, so as to obtain erythritol in the culture medium,
c) recovering erythritol from the culture medium.

12. The method according to claim 11, wherein the culture medium comprises lignocellulosic biomass, in particular straw, and/or at least one residue of dairy product production, in particular whey.

13. The method according to claim 11, wherein the culture medium comprises nitrate as a nitrogen source.

14. The method according to claim 11, wherein the culture medium comprises glycerol.

15. The method according to claim 11, wherein step b) is conducted until a concentration of erythritol in the culture medium of at least 1 g/L, preferably at least 1.5 g/L, preferably at least 2 g/L, preferably at least 2.5 g/L, preferably at least 3 g/L, is reached.

16. The method according to claim 11, wherein in step b) at least one substrate that causes osmotic stress is added to the culture medium after 14 hours of cultivation.

17. The method according to claim 16, wherein the substrate that causes osmotic stress is glycerol and/or sodium chloride.

18. Use of a genetically modified saprotroph according to claim 11 for the production of erythritol.
